(19) United States
(12) Patent Application Publication
(10) Pub. No.: US 2008/0193443 A1
(43) Pub. Date: Aug. 14, 2008

(54) Methods and compositions for the treatment of polycystic diseases

(76) Inventors: Oxana Beskrovnaya, Southboro, MA (US); Herve Husson, Boston, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/589,495

(22) Filed: Oct. 30, 2006

(51) Int. Cl. A61K 39/395; C12N 5/00; A61P 13/12; A61K 48/00

(52) U.S. Cl. 424/133.1; 435/375; 514/44; 424/130.1; 424/141.1

(57) ABSTRACT

This invention provides compositions and methods to diagnose and treat polycystic disorders by inhibiting the biological activity of a gene now correlated with appearance of this disorder. By way of illustrative only, the Connective Tissue Growth Factor (CTGF) gene is an example of such a gene. Also provided by this invention are compositions and methods to treat or ameliorate abnormal cystic lesions and diseases associated with the formation of cysts in tissue. The methods and compositions treat and ameliorate pathological cyst formation in tissue by inhibiting or augmenting gene expression or the biological activity of its gene expression product or its receptor.

### CROSS-REFERENCE TO RELATED APPLICATIONS

This application claims priority under 35 U.S.C. § 119(e) to U.S. Provisional Applications, U.S. Ser. Nos. 60/566,670 and 60/590,385, filed Apr. 29, 2004 and Jul. 22, 2004, respectively. The contents of these applications are incorporated by reference into the present disclosure.

### TECHNICAL FIELD OF THE INVENTION

This invention is related to the area of polycystic diseases and to the diagnosis and treatment of such diseases.

### BACKGROUND OF THE INVENTION

Autosomal Dominant Polycystic Kidney Disease (ADPKD) is the most common genetic renal disorder occurring in 1:1000 individuals and is characterized by focal cyst formation in all tubular segments (Friedman, J. Cystic Diseases of the Kidney, in PRINCIPLES AND PRACTICE OF MEDICAL GENETICS (A. Emery and D. Rimoin, Eds.) pp. 1002-1010, Churchill Livingston, Edinburgh, U.K. (1983); Striker & Striker (1986) Am. J. Nephrol. 6:161-164. Extrarenal manifestations include hepatic and pancreatic cysts as well as cardiovascular complications. Gabow & Grantham (1997) Polycystic Kidney Disease, in DISEASES OF THE KIDNEY (R. Schrier & C. Gottschalk, Eds.), pp. 521-560, Little Brown, Boston; Welling & Grantham (1996) Cystic Diseases of the Kidney, in RENAL PATHOLOGY (C. Tisch & B. Brenner, Eds.) pp: 1828-1863, Lippincott, Philadelphia.

To date, only PKD1 and PKD2 have been implicated as molecules responsible for these cellular abnormalities. PKD1 and PKD2 are reported to be responsible for 85% and 15% of the cases, respectively. Burn, et al. (1995) Hum. Mol. Genet. 4:575-582. Although remarkable progress toward understanding the genetics and pathophysiology of ADPKD has been made, it is still unclear how the mutations in disease-causing genes trigger cystogenesis and what other molecules play an important role in cystic phenotype.

Thus a need exists to characterize the biochemical pathway involved in the cystic phenotype and identify additional therapeutic targets. This invention satisfies this need and provides related advantages as well.

### SUMMARY OF THE INVENTION

This invention provides compositions and methods to diagnose and treat renal cystic disorders by modifying the biological activity of at least one gene identified in Tables 2 through 5, infra. As used herein, the term “renal cystic disorders” is intended to include, but not be limited to, a large group of diseases, including polycystic kidney disease, vonHippel-Lindau, tuberosclerosis, nephronophthisis, autosomal dominant polycystic kidney disease (ADPKD), autosomal recessive polycystic kidney disease (ARPKD), acquired cystic kidney disease (ACKD), and autosomal dominant polycystic liver disease (ARPKD).

By way of illustration only, the Connective Tissue Growth Factor (CTGF) gene or its expression product is an example of such a gene identified in Tables 2 through 5, infra. Accordingly, although the following discussion and examples are limited in most part to the CTGF gene and biological equivalents thereof, the invention is not so limited. The invention of this application encompasses any of the genes identified in Tables 2 through 5 as targets for therapeutic and pharmaceutical intervention; CTGF is but one member of this class of targets. Accordingly, it should be understood, although not explicitly stated, that any of the genes identified in Tables 2 through 5 can be substituted for the term “CTGF” as used herein.

In one aspect, the invention provides a method of modifying the biological activity of at least one gene identified in Tables 2 through 5 by contacting an effective amount of modifying agent or molecule with the cell or tissue in need of treatment. Suitable modifying agents for use in the method include, but are not limited to a small molecule, a ribozyme, an antisense oligonucleotide, a double stranded RNA, a double-stranded interfering RNA (iRNA), a triplex RNA, an RNA aptamer, and at least a portion of an antibody molecule that binds to the gene product and inhibits its activity. Alternatively, in some embodiments the agent binds to the receptor and initiates signaling. Examples of such antibody portions include, but are not limited to an intact antibody molecule, a single chain variable region (ScFv), a monoclonal antibody, a polyclonal antibody, a chimeric antibody, a humanized antibody or a human antibody. The antibodies can be generated in any appropriate in vitro or in vivo system, e.g., simian, murine, rat or human. Suitable antibodies are commercially available from Torrey Pines Biolabs, Inc. (Cat. No. TP 143) or Santa Cruz Biotechnology, Inc. (SC 14939). The antibody can optionally be bound to: a cytotoxic moiety, a therapeutic moiety, a detectable moiety, or an anti-cystic agent. In one aspect, the agent or molecule is isolated and then delivered.

Also provided by this invention are compositions and methods to treat or ameliorate abnormal cystic lesions and diseases associated with the formation of cysts in tissue. The methods and compositions treat and ameliorate pathological cyst formation in tissue by inhibiting, e.g., CTGF gene expression or the biological activity of its gene expression product. In some aspects, receptor activation is inhibited. In other aspects, receptor activity is initiated or augmented.

Also provided is a method of treating, inhibiting, or ameliorating the symptoms associated with Autosomal Dominant Polycystic Kidney Disease (ADPKD). The method requires delivering to a subject in need thereof an effective amount of an agent or molecule, e.g., CTGF, that modulates the activity of the CTGF gene or its expression product. In another aspect, an effective amount of an agent that inhibits the biological activity of the CTGF receptor is delivered to the subject. U.S. Pat. No. 6,555,322 discloses cDNA sequence encoding a receptor as well as its amino acid sequence. In one aspect, the agent or molecule is isolated and then delivered. In another aspect, where gene underexpression contributes to the disease or pathology, delivery of a gene or polypeptide that augments expression is delivered. Such agents are known in the art and include, but are not limited to polynucleotides encoding the peptides or the polypeptides themselves.

This invention also provides methods for aiding in the diagnosis of cystic abnormalities present in a tissue by detecting the expression level of the gene or its expression product. The method can be used for aiding in the diagnosis of ADPKD-associated renal cysts and cystic abnormalities in other organs, including the liver, pancreas, spleen and ovaries, that are commonly found in ADPKD. Additionally, by detecting overexpression or underexpression of the protein or polynucleotide prior to abnormal cyst formation, one can predict a predisposition to ADPKD and provide early diagnosis and/or treatment.

Further provided are kits for carrying out the diagnostic and prognostic methods. The kits contain compositions used in these methods and instructions for their use.

This invention also provides compositions for use in the therapeutic and diagnostic methods. In one aspect, the composition comprises a molecule containing an antibody variable region which specifically binds to a CTGF protein (e.g., SEQ ID NO: 2) or its receptor so that binding is inhibited and/or blocked and the receptor is not activated or activation of the receptor is inhibited. The molecule can be, for example, an intact antibody molecule, a single chain variable region (ScFv), a monoclonal antibody, a chimeric or a humanized antibody. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. The molecule can optionally be bound to: a cytotoxic moiety, a therapeutic moiety, a detectable moiety or an anti-cystic agent.

In another aspect, the invention provides nucleic acid molecules that inhibit the expression of the CTGF gene or the receptor to which CTGF protein binds, or the gene that encodes the receptor. These nucleic acids are described herein and include, but are not limited to a ribozyme, an antisense oligonucleotide, a double stranded RNA, iRNA, a triplex RNA or an RNA aptamer. In one aspect, the nucleic acid is delivered in an isolated form. The nucleic acid can be isolated from an animal or alternatively, recombinantly produced in any suitable recombinant system, e.g., bacterial, yeast, baculoviral or mammalian.

In yet another aspect, the invention provides nucleic acid molecules that enhance, support, augment or increase expression of the gene or, its transcription and/or translation product or any ligand which activates the receptor. These nucleic acids are described herein and include, but are not limited to a ribozyme, an antisense oligonucleotide, a double stranded RNAs, iRNA, a triplex RNA or an RNA aptamer. In one aspect, the nucleic acid is delivered in an isolated form. The nucleic acid can be isolated from an animal or alternatively, recombinantly produced in any suitable recombinant system, e.g., bacterial, yeast, baculoviral or mammalian.

Yet another aspect of the invention is a method to identify a CTGF binding ligand involved in CTGF-associated cyst formation. A test compound or agent such as an antibody or antibody derivative or variant is contacted with a CTGF protein or fragment thereof in a suitable sample under conditions that favor the formation of binding to CTGF or its receptor. Receptor-binding or CTGF-binding, if it occurred, is then detected.

In one aspect, the therapeutic and diagnostic agents are used in combination with other agents. Co-administration of these agents or molecules with other agents or therapies can provide unexpected synergistic therapeutic benefit. In the co-administration methods, the agents or molecules are also useful in reducing deleterious side-effects of known therapies and therapeutic agents, as well as yet to be discovered therapies and therapeutic agents, by decreasing dosage. In one aspect, the use of operative combinations is contemplated to provide therapeutic combinations requiring a lower total dosage of each component than may be required when each individual therapeutic method, compound or drug is used alone, thereby reducing adverse side effects. Thus, the present invention also includes methods involving co-administration of the compounds described herein with one or more additional active agents or methods. Indeed, it is a further aspect of this invention to provide methods for enhancing other therapies and/or pharmaceutical compositions by co-administering a compound of this invention. In co-administration procedures, the agents may be administered concurrently or sequentially. In one embodiment, the compounds described herein are administered prior to the other active agent(s), therapy or therapies. The pharmaceutical formulations and modes of administration may be any of those described herein or known to those of skill in the art.

A still further embodiment of the invention is a method to identify candidate drugs to treat cystic lesions by contacting cells which express the CTGF gene or its receptor with a test compound or agent. A test compound is identified as a candidate drug for treating cystic abnormalities if it increases or decreases expression of the CTGF gene or the gene encoding its receptor. Expression can be detected and quantified by any method known in the art, e.g., by hybridization of mRNA of the cells or tissue to a nucleic acid probe which is complementary to CTGF mRNA or receptor mRNA, where appropriate. Test compounds or agents which decrease expression are identified as candidates for treating abnormal CTGF cyst formation.

Applicants also provide kits for determining whether a pathological cell or a patient will be suitably treated by one or more of the therapies described herein. Additionally, kits for performance of the assays are provided. These kits contain at least one composition of this invention and instructions for use.

### BRIEF DESCRIPTION OF THE FIGURES

FIG. 1 shows the effect of an anti-CTGF antibody in an in vitro model of cystogenesis.

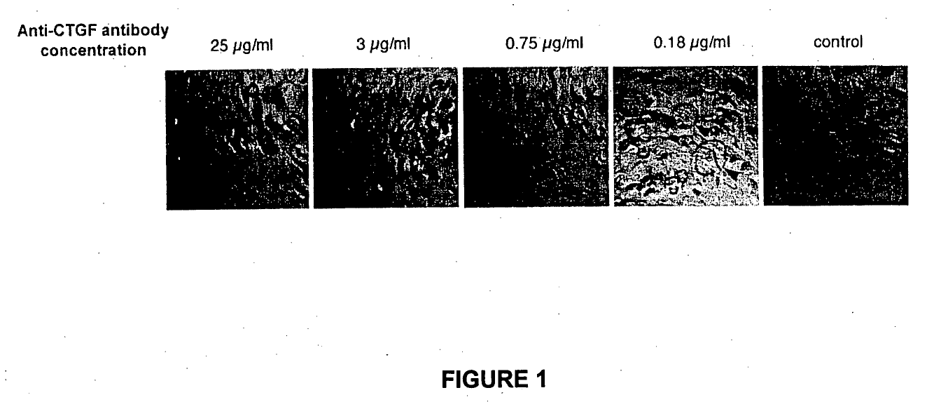

FIG. 2 shows that CTGF protein is expressed in jck mouse kidneys.

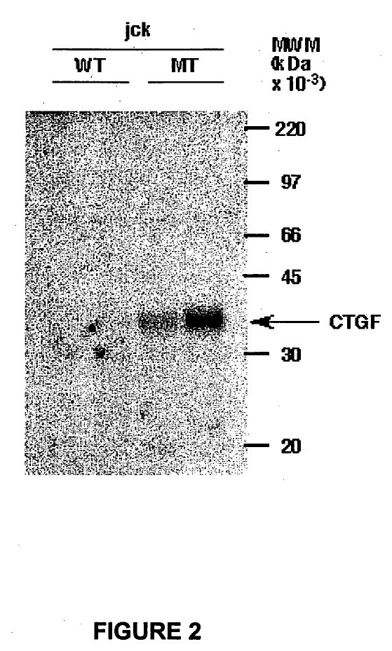

FIG. 3 is a 2D SDS gel profiling of cyst fluid from ADPKD patient.

### BRIEF DESCRIPTION OF THE TABLES

Table 1A is a summary of SAGE libraries screened. It is a summary of total tags sequenced and unique tags. Table 1B summarizes CTGF expression in normal and cystic kidneys.

Table 2 identifies the top 20 up- and down-regulated genes in cystic liver (CL) (SEQ ID NOS: 3-43, respectively, in order of appearance).

The 20 most down- (top panel) or up-regulated (bottom panel) tags (10 bases long) along with their counts in normal liver (NL) or cystic liver (CL) epithelial libraries, Genebank accession number, gene denomination and HUGO assignment are presented. The 11^{th} base of the Tag is presented to help discriminate between genes when 10 base-Tag had several Unigene matches.

Table 3 identifies the top 20 up- and down-regulated genes in cystic kidney (CK) (SEQ ID NOS: 44-83, respectively, in order of appearance). The 20 most down- or up-regulated tags along with their counts in normal kidney (NK) or cystic kidney (CK) are represented as for the ones presented in Table 2.

Table 4 identifies the up-regulated genes >5× common to liver and kidney epithelia (SEQ ID NOS: 84-111, respectively, in order of appearance). Common genes up-regulated in CK and CL are presented with the 10 base Tag sequence, the 11 h base, CL/NL and CK/NK ratios, Genebank accession number, gene description and corresponding HUGO name.

Table 5A identifies functional groups of genes up-regulated in CL (SEQ ID NOS: 112-155, respectively, in order of appearance). Table 5B identifies functional groups of genes up-regulated in CK (SEQ ID NOS: 156-193, respectively, in order of appearance).

### Modes for Carrying Out the Invention

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

As used herein, certain terms have the following defined meanings.

### DEFINITIONS

The practice of the present invention will employ, unless otherwise indicated, conventional techniques of immunology, molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

The term “polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The term “isolated” means separated from constituents, cellular and otherwise, in which the polynucleotide, peptide, polypeptide, protein, antibody, or fragments thereof, are normally associated with in nature. In one aspect of this invention, an isolated polynucleotide is separated from the 3′ and 5′ contiguous nucleotides with which it is normally associated with in its native or natural environment, e.g., on the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

The terms “polynucleotide” and “oligonucleotide” are used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for guanine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated. Methods of isolating larger fragment sequences are known to those of skill in the art, some of which are described herein.

A “gene product” or “expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” refers to a juxtaposition wherein the elements are in an arrangement allowing them to function.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky, Curr. Opin. Biotechnol. (1999) 5:434-439 and Ying, et al., Nat. Med. (1999) 5(7):823-827. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads are easy to grow and do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka, Proc. Natl. Acad. Sci. USA (1984) 81:6466-6470 and Lebkowski, et al., Mol. Cell. Biol. (1988) 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

An expression “database” denotes a set of stored data that represent a collection of sequences, which in turn represent a collection of biological reference materials.

The term “cDNAs” refers to complementary DNA, that is mRNA molecules present in a cell or organism made in to cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

“Differentially expressed” as applied to a gene, refers to the differential production of the mRNA transcribed from the gene or the protein product encoded by the gene. A differentially expressed gene may be overexpressed or underexpressed as compared to the expression level of a normal or control cell. In one aspect, it refers to a differential that is 2.5 times, or alternatively 5 times, or alternatively 10 times higher or lower than the expression level detected in a control sample. The term “differentially expressed” also refers to nucleotide sequences in a cell or tissue which are expressed where silent in a control cell or not expressed where expressed in a control cell.

As used herein, “solid phase support” or “solid support”, used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, California).

A polynucleotide also can be attached to a solid support for use in high throughput screening assays. International PCT Application No. WO 97/10365, for example, discloses the construction of high density oligonucleotide chips. See also, U.S. Pat. Nos. 5,405,783; 5,412,087; and 5,445,934. Using this method, the probes are synthesized on a derivatized glass surface also known as chip arrays. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell. “Overexpression” as applied to a gene, refers to the overproduction of the mRNA transcribed from the gene or the protein product encoded by the gene, at a level that is 2.5 times higher, or alternatively 5 times higher, or alternatively 10 times higher than the expression level detected in a control sample.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

“Suppressing” cell growth means any or all of the following states: slowing, delaying, and stopping tumor growth, as well as tumor shrinkage. Cell and tissue growth can be assessed by any means known in the art, including but not limited to measuring cyst size, determining whether cells are proliferating using a ³H-thymidine incorporation assay, or counting cells.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “effective amount” is an amount sufficient to effect beneficial or desired results. An effective amount can be administered in one or more administrations, applications or dosages.

A “subject,” “individual” or “patient” is used interchangeably herein, which refers to a vertebrate, preferably a mammal, more preferably a human. Mammals include, but are not limited to, murines, rats, simians, humans, farm animals, sport animals, and pets.

A “control” is an alternative subject or sample used in an experiment for comparison purpose. A control can be “positive” or “negative”. For example, where the purpose of the experiment is to determine a correlation of an altered expression level of a gene with a particular type of cancer or pathology, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

### Therapeutic Methods

This invention provides methods for treating and/or ameliorating the symptoms associated with cystic abnormalities present in a tissue. In one aspect, the cysts are a manifestation of Autosomal Dominant Polycystic Kidney Disease (ADPKD). The major manifestation of the disorder is the progressive cystic dilation of renal tubules which ultimately leads to renal failure in half of affected individuals. U.S. Pat. No. 5,891,628 and Gabow, P. A., Am. J. Kidney Dis. (1990) 16:403-413. ADPKD-associated renal cysts may enlarge to contain several liters of fluid and the kidneys usually enlarge progressively causing pain. Other abnormalities such as hematuria, renal and urinary infection, renal tumors, salt and water imbalance and hypertension frequently result from the renal defect. Cystic abnormalities in other organs, including the liver, pancreas, spleen and ovaries are commonly found in ADPKD. Massive liver enlargement occasionally causes portal hypertension and hepatic failure. Cardiac valve abnormalities and an increased frequency of subarachnoid and other intracranial hemorrhage have also been observed in ADPKD. U.S. Pat. No. 5,891,628. Although studies of kidneys from ADPKD patients have demonstrated a number of different biochemical, structural and physiological abnormalities, the disorder's underlying causative biochemical defect remains unknown. Biochemical abnormalities which have been observed have involved protein sorting, the distribution of cell membrane markers within renal epithelial cells, extracellular matrix, ion transport, epithelial cell turnover, and epithelial cell proliferation. The most carefully documented of these findings are abnormalities in the composition of tubular epithelial cells, and a reversal of the normal polarized distribution of cell membrane proteins, such as the Na⁺/K⁺ ATPase. Carone, F. A. et al., Lab. Inv. (1994) 70:437-448. Thus, this invention provides methods for inhibiting, reducing or ameliorating the above-noted biochemical, structural and physiological abnormalities related to ADPKD.

The method requires delivering to the tissue in need thereof an effective amount of an agent or molecule that modifies (inhibits or augments) expression of a gene identified in Tables 2 through 5 or its expression product, in affected tissue. In one aspect, Applicants have discovered quite unexpectedly, that overexpression of the CTGF gene in tissue is related to cystic abnormalities and that downregulation of the gene or its expression product treats or ameliorates the symptoms associated with cystic abnormalities. Inhibiting the binding of CTGF to its receptor also treats or ameliorates symptoms associated with cystic abnormalities.

Pathologically, CTGF has previously been postulated to be involved in conditions in which there is an overgrowth of connective tissue cells, such as systemic sclerosis, cancer, fibrotic conditions, and atherosclerosis. The primary biological activities of CTGF polypeptide are reported to be related to its mitogenicity, or ability to stimulate target cells to proliferate and its role in the synthesis of the extracellular matrix. The ultimate result of this mitogenic activity in vivo, is the growth of targeted tissue. CTGF also is reported to possess chemotactic activity, which is the chemically induced movement of cells as a result of interaction with particular molecules. Elevated levels of CTGF are found in fibrotic lesions and suggested to be functionally involved in the development of fibrotic diseases and wound healing. Chih-Chiun, C. et al., J. Biol. Chem. (2001) 276(13):10443-10452; Hahn, A. et al., J. Biol. Chem. (2000) 275(48):3749-3735. Compositions and methods for modulation of the growth factor in connection with proliferative diseases also have been previously reported. See, U.S. Patent Publ. Docs. US 2002/0115156 A1 and US 2002/0142353 A1; U.S. Pat. Nos. 6,555,322 B1; 6,358,741 B1; 6,348,329; 5,783,187 B1; 5,408,040, and International PCT Application Nos. WO 00/35939; WO 01/15729 A1; WO 00/13706; WO 96/38168 and WO 96/38172.

CTGF is a cysteine-rich monomeric peptide of Mᵣ 38,000. It is a member of the CCN family of growth regulators which includes the mouse (also known as fisp-12 or βIG-M2) and human CTGF, Cyr61 (mouse), Cef10 (chicken), and Nov (chicken). Based on sequence comparisons, it has been suggested that the members of this family all have a modular structure, consisting of (1) an insulin-like growth factor domain responsible for binding, (2) a von Willebrand factor domain responsible for complex formation, (3) a thrombospondin type I repeat, possibly responsible for binding matrix molecules, and (4) a C-terminal module found in matrix proteins, postulated to be responsible for receptor binding.

CCN is the acronym for an emerging family of regulatory proteins which are reported to be involved in the regulation of cell proliferation, chemotaxis, angiogenesis and the formation of the extracellular matrix. Lin, C. G. et al., J. Biol. Chem. (2003) 278(26):24200-24208; Lafont, J. et al., J. Biol. Chem. (2002) 277(43):41220-41229; Li, C. L. et al., J. Clin. Pathol. (2002) 55:250-261; Manara, M. C. et al., Am. J. Pathol. (2002) 160(3):849-859. The family comprises both positive and negative regulators that share a common multi-modular organization. See, generally, Perbal, B., Mol. Pathol. (2001) 54:103-104, and references cited therein.

The cDNA for human CTGF (hCTGF) has been reported to contain an open reading frame of 1047 nucleotides with an initiation site at position 130 and a TGA termination site at position 1177. The cDNA encodes a peptide of 349 amino acids. See, U.S. Patent Publ. US 2002/0115156A1. The cDNA sequence is also available at GenBank No.: NM_{—}001901, which is also reproduced as SEQ ID NO: 1. The gene is reported to contain 2312 nucleotides with the open reading frame represented by nucleotides 146 through 1195. The 572 amino acid polypeptide expressed from this sequence is available under GenBank No.: NP_{—}001892.1, which is also reproduced as SEQ ID NO: 2.

The cDNA sequence for rat CTGF is reported to contain an open reading frame of 2350 nucleotides with an initiation site at position 212 and a TAA termination site at position 1353 and encodes a peptide of 346 amino acids. See, paragraph 28 of U.S. Published Patent Doc. US 2002/0115156A1.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

“Treating” also covers any treatment of a disorder in a mammal, and includes: (a) preventing a disorder from occurring in a subject that may be predisposed to a disorder, but has not yet been diagnosed as having it; (b) inhibiting a disorder, i.e., arresting its development; or (c) relieving or ameliorating the disorder, e.g., cause regression of the disorder, e.g., ADPKD.

As used herein, to “treat” includes systemic amelioration of the symptoms associated with the pathology and/or a delay in onset of symptoms. Clinical and sub-clinical evidence of “treatment” will vary with the pathology, the individual and the treatment.

Overexpression or in some instances, underexpression, of a gene identified in Tables 2 through 5, results in a pathological state in cells and/or tissue which are then suitably treated by the methods of this invention. These cells or tissue are identified by any method known in the art that allows for the identification of differential expression of the gene or its expression product. Exemplary methods are described herein.

Therapeutic agents can be administered to suitable cells, tissues or to subjects as well as or in addition to individuals susceptible to or at risk of developing cystic abnormalities. When the agent is administered to a subject such as a mouse, a rat or a human patient, the agent can be added to a pharmaceutically acceptable carrier and systemically or topically administered to the subject. To determine patients that can be beneficially treated, a regression of the cyst can be assayed. Therapeutic amounts can be empirically determined and will vary with the pathology being treated, the subject being treated and the efficacy and toxicity of the therapy.

Administration in vivo can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art.

The agents and compositions of the present invention can be used in the manufacture of medicaments and for the treatment of humans and other animals by administration in accordance with conventional procedures, such as an active ingredient in pharmaceutical compositions.

An agent of the present invention can be administered for therapy by any suitable route including nasal, topical (including transdermal, aerosol, buccal and sublingual), parental (including subcutaneous, intramuscular, intravenous and intradermal) and pulmonary. It will also be appreciated that the preferred route will vary with the condition and age of the recipient, and the disease being treated.

The polynucleotides useful for the methods of this invention can be replicated using PCR. PCR technology is the subject matter of U.S. Pat. Nos. 4,683,195; 4,800,159; 4,754,065; and 4,683,202 and described in PCR: THE POLYMERASE CHAIN REACTION (Mullis et al. eds, Birkhauser Press, Boston (1994)) and references cited therein.

Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

RNA can be obtained by first inserting a DNA polynucleotide into a suitable host cell. The DNA can be inserted by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook et al. (1989) supra. For instance, mRNA can be isolated using various lytic enzymes or chemical solutions according to the procedures set forth in Sambrook, et al. (1989) supra or extracted by nucleic-acid-binding resins following the accompanying instructions provided by manufactures.

Antisense nucleic acids are DNA or RNA molecules that are complementary to at least a portion of a specific transcript RNA molecule. In the cell, the antisense nucleic acids hybridize to the corresponding transcript RNA, forming a double-stranded molecule thereby interfering with the translation of the mRNA, since the cell will not translate a mRNA that is double-stranded. Antisense oligomers of about 15 nucleotides are preferred, since they are easily synthesized and are less likely to cause problems than larger molecules. The use of antisense methods to inhibit the in vitro translation of genes is known in the art. Marcus-Sakura, Anal. Biochem. (1988) 172:289 and De Mesmaeker, et al., Curr. Opin. Struct. Biol. (1995) 5:343-355. The information disclosed in these publications and known to those of skill in the art, in combination with Applicants' specification, enables one of skill in the art to make and use antisense DNA or RNA molecules as therapeutic agents.

Use of an oligonucleotide to stall transcription is known as the triplex strategy since the oligomer winds around double-helical DNA, forming a three-strand helix. Triplex compounds are designed to recognize a unique site on a chosen gene. Maher, et al., Antisense Res. and Dev. (1991) 1(3):227; Helene, C., Anticancer Drug Design (1991) 6(6):569.

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it. A major advantage of this approach is that, because they are sequence-specific, only mRNAs with particular sequences are inactivated.

U.S. Pat. No. 6,458,559 discloses how to make and use RNA aptamer molecules to inhibit gene expression. The information disclosed in this patent, in combination with the Applicants' specification, enables one of skill in the art to make and use aptamers as CTGF inhibitory molecules.

U.S. Published Patent Doc. US 20030180744 discloses methods to make and use high affinity oligonucleotide ligands to growth factors. The information disclosed in this published application, in combination with the Applicants' specification, enables one of skill in the art to make and use oligonucleotide ligands as therapeutic molecules.

U.S. Published Patent Doc. US 20030051263 discloses a process for introducing a double stranded RNA into a living cell to inhibit gene expression of a target gene in that cell. Inhibition is sequence-specific in that the nucleotide sequences of the duplex region of the RNA and of a portion of the target gene are identical. The information disclosed in this published application, in combination with the Applicants' specification, enables one of skill in the art to make and use double stranded RNA molecules as therapeutic agents. See, e.g., Elbashir, S. M. et al., Nature (2001) 411:494.

When the agent is a nucleic acid, it can be added to the cell cultures by methods known in the art, which include, but are not limited to calcium phosphate precipitation, microinjection or electroporation. They can be added alone or in combination with a suitable carrier, e.g., a pharmaceutically acceptable carrier such as phosphate buffered saline. Alternatively or additionally, the nucleic acid can be incorporated into an expression or insertion vector for incorporation into the cells. Vectors that contain both a promoter and a cloning site into which a polynucleotide can be operatively linked are known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression. Examples of vectors are viruses, such as baculovirus and retrovirus, bacteriophage, adenovirus, adeno-associated virus, cosmid, plasmid, fungal vectors and other recombination vehicles typically used in the art which have been described for expression in a variety of eukaryotic and prokaryotic hosts, and may be used for gene therapy as well as for simple protein expression.

Among these are several non-viral vectors, including DNA/liposome complexes, and targeted viral protein DNA complexes. To enhance delivery to a cell, the nucleic acid or proteins of this invention can be conjugated to antibodies or binding fragments thereof which bind cell surface antigens. Liposomes that also comprise a targeting antibody or fragment thereof can be used in the methods of this invention. This invention also provides the targeting complexes for use in the methods disclosed herein.

Polynucleotides are inserted into vector genomes using methods known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA. Other means are known and available in the art.

This invention also provides isolated polypeptides encoded by a gene identified in Tables 2 through 5, e.g., the CTGF gene. In one aspect, the CTGF polypeptide has the amino acid sequence shown in SEQ ID NO: 2. In another aspect, the polypeptide is modified by substitution with conservative amino acids. In yet a further aspect, the polypeptide has the same function as the polypeptide of SEQ ID NO: 2 as determined using the examples set forth below and are identified by having more than 80%, or alternatively, more than 85%, or alternatively, more than 90%, or alternatively, more than 95%, or alternatively more than 97%, or alternatively, more than 98 or 99% sequence homology to SEQ ID NO: 2 as determined by sequence comparison programs such as BLAST run under appropriate conditions. In one aspect, the program is run under default parameters. Further provided are active fragments of these embodiments.

The peptides used in accordance with the method of the present invention can be obtained in any one of a number of conventional ways. For example, peptides can be prepared by chemical synthesis using standard techniques. Particularly convenient are the solid phase peptide synthesis techniques. Automated peptide synthesizers are commercially available, as are the reagents required for their use.

In one embodiment, isolated peptides of the present invention can be synthesized using an appropriate solid state synthetic procedure. Steward and Young, eds. (1968) SOLID PHASE PEPTIDE SYNTHESIS, Freemantle, San Francisco, Calif. One method is the Merrifield process. Merrifield, Recent Progress in Hormone Res. (1967) 23:451. Once an isolated peptide of the invention is obtained, it may be purified by standard methods including chromatography (e.g., ion exchange, affinity, and sizing column chromatography), centrifugation, differential solubility, or by any other standard technique for protein purification. For immunoaffinity chromatography, an epitope may be isolated by binding it to an affinity column comprising antibodies that were raised against that peptide, or a related peptide of the invention, and were affixed to a stationary support.

Alternatively, affinity tags such as hexa-His (Invitrogen), Maltose binding domain (New England Biolabs), influenza coat sequence (Kolodziej et al., Methods Enzymol. (1991) 194:508-509), and glutathione-S-transferase can be attached to the peptides of the invention to allow easy purification by passage over an appropriate affinity column. Isolated peptides can also be physically characterized using such techniques as proteolysis, nuclear magnetic resonance, and x-ray crystallography.

Alternatively, the polynucleotides can be replicated using PCR or gene cloning techniques. Thus, this invention also provides a polynucleotide of this invention operatively linked to elements necessary for the transcription and/or translation of these polynucleotides in host cells. In one aspect, the polynucleotide is a component of a gene delivery vehicle for insertion into the host cells. The means by which the cells may be transformed with the expression construct includes, but is not limited to, microinjection, electroporation, transduction, transfection, lipofection, calcium phosphate particle bombardment mediated gene transfer or direct injection of nucleic acid sequences or other procedures known to one skilled in the art (Sambrook et al. (1989) supra). For various techniques for transforming mammalian cells, see, e.g., Keown et al., Methods in Enzymology (1990) 185:527-537.

Host cells include eukaryotic and prokaryotic cells, such as bacterial cells, yeast cells, simian cells, murine cells and human cells. The cells can be cultured or recently isolated from a subject. The host cells are cultured under conditions necessary for the recombinant production of the polypeptide or recombinant replication of the polynucleotides. Recombinantly produced polynucleotides and/or polynucleotides are further provided herein.

Also included within the scope of the invention are polypeptides that are differentially modified during or after translation, e.g., by phosphorylation, glycosylation, crosslinking, acetylation, proteolytic cleavage, linkage to an antibody molecule, membrane molecule or other ligand. Ferguson et al., Ann. Rev. Biochem. (1988) 57:285-320. This is achieved using various chemical methods or by expressing the polynucleotides in different host cells, e.g., bacterial, mammalian, yeast, or insect cells.

Also provided by this invention are peptide fragments, e.g., immunogeneic or antigenic portions, alone or in combination with a carrier. An antigenic peptide epitope of the invention can be used in a variety of formulations, which may vary depending on the intended use.

An antigenic peptide epitope of the invention can be covalently or non-covalently linked (complexed) to various other molecules, the nature of which may vary depending on the particular purpose. For example, a peptide of the invention can be covalently or non-covalently complexed to a macromolecular carrier, including, but not limited to, natural and synthetic polymers, proteins, polysaccharides, poly(amino acid), polyvinyl alcohol, polyvinyl pyrrolidone, and lipids. A peptide can be conjugated to a fatty acid, for introduction into a liposome. U.S. Pat. No. 5,837,249. A synthetic peptide of the invention can be complexed covalently or non-covalently with a solid support, a variety of which are known in the art. An antigenic peptide epitope of the invention can be associated with an antigen-presenting matrix with or without co-stimulatory molecules, as described in more detail below.

Examples of protein carriers include, but are not limited to, superantigens, serum albumin, tetanus toxoid, ovalbumin, thyroglobulin, myoglobulin, and immunoglobulin.

Peptide-protein carrier polymers may be formed using conventional crosslinking agents such as carbodiimides. Examples of carbodiimides are 1-cyclohexyl-3-(2-morpholinyl-(4-ethyl) carbodiimide (CMC), 1-ethyl-3-(3-dimethyaminopropyl) carbodiimide (EDC) and 1-ethyl-3-(4-azonia-44-dimethylpentyl) carbodiimide.

Examples of other suitable crosslinking agents are cyanogen bromide, glutaraldehyde and succinic anhydride. In general, any of a number of homobifunctional agents including a homobifunctional aldehyde, a homobifunctional epoxide, a homobifunctional imidoester, a homobifunctional N-hydroxysuccinimide ester, a homobifunctional maleimide, a homobifunctional alkyl halide, a homobifunctional pyridyl disulfide, a homobifunctional aryl halide, a homobifunctional hydrazide, a homobifunctional diazonium derivative and a homobifunctional photoreactive compound may be used. Also included are heterobifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homobifunctional crosslinking agents include the bifunctional N-hydroxysuccinimide esters dithiobis(succinimidylpropionate), disuccinimidyl suberate, and disuccinimidyl tartarate; the bifunctional imidoesters dimethyl adipimidate, dimethyl pimelimidate, and dimethyl suberimidate; the bifunctional sulfhydryl-reactive crosslinkers 1,4-di-[3′-(2′-pyridyldithio)propion-amido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether, the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri(2-chloroethyl)amine, respectively.

Examples of other common heterobifunctional cross-linking agents that may be used to effect the conjugation of proteins to peptides include, but are not limited to, SMCC succinimidyl-4-(N-maleimidomethyl)cyclohexane-1-carboxylate), MBS (m-maleimidobenzoyl-N-hydroxysuccinimide ester), SIAB (N-succinimidyl(4-iodoacteyl)aminobenzoate), SMPB (succinimidyl-4-(p-maleim idophenyl)butyrate), GMBS (N-(γ-maleimidobutyryloxy)succinimide ester), MPBH (4-(4-N-maleimidopohenyl) butyric acid hydrazide), M2C2H (4-(N-maleimidomethyl) cyclohexane-1-carboxyl-hydrazide), SMPT (succinimidyloxycarbonyl-α-methyl-α-(2-pyridyldithio)toluene), and SPDP (N-succinimidyl 3-(2-pyridyldithio)propionate).

Crosslinking may be accomplished by coupling a carbonyl group to an amine group or to a hydrazide group by reductive amination.

Peptides of the invention also may be formulated as non-covalent attachment of monomers through ionic, adsorptive, or biospecific interactions. Complexes of peptides with highly positively or negatively charged molecules may be done through salt bridge formation under low ionic strength environments, such as in deionized water. Large complexes can be created using charged polymers such as poly-(L-glutamic acid) or poly-(L-lysine) which contain numerous negative and positive charges, respectively. Adsorption of peptides may be done to surfaces such as microparticle latex beads or to other hydrophobic polymers, forming non-covalently associated peptide-superantigen complexes effectively mimicking crosslinked or chemically polymerized protein. Finally, peptides may be non-covalently linked through the use of biospecific interactions between other molecules. For instance, utilization of the strong affinity of biotin for proteins such as avidin or streptavidin or their derivatives could be used to form peptide complexes. These biotin-binding proteins contain four binding sites that can interact with biotin in solution or be covalently attached to another molecule. Wilchek, Anal Biochem. (1988) 171:1-32. Peptides can be modified to possess biotin groups using common biotinylation reagents such as the N-hydroxysuccinimidyl ester of D-biotin (NHS-biotin) which reacts with available amine groups on the protein. Biotinylated peptides then can be incubated with avidin or streptavidin to create large complexes. The molecular mass of such polymers can be regulated through careful control of the molar ratio of biotinylated peptide to avidin or streptavidin.

Also provided by this application are the peptides and polypeptides described herein conjugated to a detectable agent for use in the diagnostic methods. For example, detectably labeled peptides and polypeptides can be bound to a column and used for the detection and purification of antibodies. They also are useful as immunogens for the production of antibodies, as described below.

The peptides of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to, Freund's Complete and Incomplete, mineral salts and polynucleotides.

The proteins and polypeptides of this invention can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this invention also provides a process for chemically synthesizing the proteins of this invention by providing the sequence of the protein and reagents, such as amino acids and enzymes and linking together the amino acids in the proper orientation and linear sequence.

One can determine if the object of the method, i.e., reversal of the pathological state of the cell or tissue, has been achieved by a reduction of cell division, differentiation of the cell or a reduction in CTGF overexpression. Cellular differentiation can be monitored by histological methods or by monitoring for the presence or loss of certain cell surface markers. The reversal of pathological state in humans can be measured by the reduction in cystic (or renal) volume, using NMR.

The method can also be practiced by delivering to the affected tissue an effective amount of therapeutic agent such as a blocking or inhibitory antibody or derivative thereof or small molecules. An exemplary antibody is described infra. These can be delivered alone or in combination with a carrier such as a pharmaceutically acceptable carrier.

Using the proteins according to the invention, one of ordinary skill in the art can readily generate additionally antibodies which specifically bind to the protein or fragments thereof. Such antibodies can be monoclonal or polyclonal. They can be chimeric, humanized, or totally human. Any functional fragment or derivative of an antibody can be used including Fab, Fab′, Fab2, Fab′2, and single chain variable regions. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. So long as the fragment or derivative retains specificity of binding for the protein or fragment thereof it can be used. Antibodies can be tested for specificity of binding by comparing binding to appropriate antigen to binding to irrelevant antigen or antigen mixture under a given set of conditions. If the antibody binds to the appropriate antigen at least 2, 5, 7, and preferably 10 times more than to irrelevant antigen or antigen mixture then it is considered to be specific.

Techniques for making such partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. See for example, Russel, N. D. et al., Infection and Immunity (2000) April 2000:1820-1826; Gallo, M. L. et al., European J. of Immun. (2000) 30:534-540; Green, L. L., J. of Immun. Methods (1999) 231:11-23; Yang, X-D et al., J. of Leukocyte Biology (1999A) 66:401-410; Yang, X-D, Cancer Research (1999B) 59(6):1236-1243; Jakobovits, A., Advanced Drug Delivery Reviews (1998) 31:33-42; Green, L. and Jakobovits, A., J. Exp. Med. (1998) 188(3):483-495; Jakobovits, A., Exp. Opin. Invest. Drugs (1998) 7(4):607-614; Tsuda, H. et al., Genomics (1997) 42:413-421; Sherman-Gold, R., Genetic Engineering News (1997) 17(14); Mendez, M. et al., Nature Genetics (1997) 15:146-156; Jakobovits, A., WEIR'S HANDBOOK OF EXPERIMENTAL IMMUNOLOGY, THE INTEGRATED IMMUNE SYSTEM VOL. IV, (1996) 194.1-194.7; Jakobovits, A., Current Opinion in Biotechnology (1995) 6:561-566; Mendez, M. et al., Genomics (1995) 26:294-307; Jakobovits, A., Current Biology (1994) 4(8):761-763; Arbones, M. et al., Immunity (1994) 1(4):247-260; Jakobovits, A., Nature (1993) 362(6417):255-258; Jakobovits, A. et al., Proc. Natl. Acad. Sci. USA (1993) 90(6):2551-2555; Kucherlapati, et al., U.S. Pat. No. 6,075,181.

Antibodies can also be made using phage display techniques. Such techniques can be used to isolate an initial antibody or to generate variants with altered specificity or avidity characteristics. Single chain Fv can also be used as is convenient. They can be made from vaccinated transgenic mice, if desired. Antibodies can be produced in cell culture, in phage, or in various animals, including but not limited to cows, rabbits, goats, mice, rats, hamsters, guinea pigs, sheep, dogs, cats, monkeys, chimpanzees, apes, etc.

Antibodies can be labeled with a detectable moiety such as a radioactive atom, a chromophore, a fluorophore, or the like. Such labeled antibodies can be used for diagnostic techniques, either in vivo, or in an isolated test sample. Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; Iy207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

### Diagnostic Methods

In one aspect, this invention provides methods for aiding in the diagnosis of cystic abnormalities present in a tissue. The pathological state of the cell or tissue is identified by differential expression of the CTGF gene, the gene for its receptor or their expression products. In general, gene expression is determined by noting the amount (if any, e.g., altered) expression of the gene in the test system, e.g., differential expression is determined by an increase or in some aspects a decrease, by at least 1.5 fold, 2.5 fold, or alternatively at least 5 fold, or alternatively 10 fold, in the level of a mRNA transcribed from the gene. In a separate embodiment, augmentation of the level of the polypeptide or protein encoded by the gene is indicative of the presence of the pathological condition of the cell. The method can be used for aiding in the diagnosis of ADPKD-associated renal cysts and cystic abnormalities in other organs, including the liver, pancreas, spleen and ovaries that are commonly found in ADPKD.

Additionally, by detecting differential expression of protein or gene prior to abnormal cyst formation, one can predict a predisposition to cystic abnormalities and/or provide early diagnosis and treatment.

Cell or tissue samples used for this invention encompass body fluid, solid tissue samples, tissue cultures or cells derived there from and the progeny thereof, and sections or smears prepared from any of these sources, or any other samples that may contain a cell having differential expression. A preferred sample is one that is prepared from a subject's renal tubules.

### Diagnostic Methods Utilizing Recombinant DNA Technology and Bioinformatics

In one aspect, the invention provides compositions and methods for diagnosing or monitoring cystic abnormalities, such as those associated with ADPKD disease by determining the expression level of the CTGF gene or its receptor and correlating the determined level of expression with said disease or its progression. Various methods are known for quantifying the expression of a gene of interest and include but are not limited to hybridization assays (Northern blot analysis) and PCR based hybridization assays.

In assaying for an alteration in mRNA level, the nucleic acid contained in a sample is first extracted according to a standard method in the art. For instance, mRNA can be isolated using various lytic enzymes or chemical solutions according to the procedures set forth in Sambrook et al. (1989), supra or extracted by nucleic-acid-binding resins following the accompanying instructions provided by the manufacturers. As an example, the CTGF mRNA contained in the extracted nucleic acid sample is then detected by hybridization (e.g., Northern blot analysis) and/or amplification procedures using nucleic acid probes and/or primers, respectively, according to standard procedures.

Nucleic acid molecules having at least 10 nucleotides and exhibiting sequence complementarity or homology to the CTGF can be used as CTGF hybridization probes or CTGF PCR primers in the diagnostic methods. It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. For example, a probe useful for detecting CTGF mRNA is at least about 80% identical to the homologous region of comparable size contained in a previously identified sequence, e.g., see SEQ ID NO: 1. Alternatively, the probe is at least 85% or even at least 90% identical to the corresponding gene sequence after alignment of the homologous region. The total size of fragment, as well as the size of the complementary stretches, will depend on the intended use or application of the particular nucleic acid segment. Smaller fragments of the gene will generally find use in hybridization embodiments, wherein the length of the complementary region may be varied, such as between about 10 and about 100 nucleotides, or even full length according to the complementary sequences one wishes to detect.

Nucleotide probes having complementary sequences over stretches greater than about 10 nucleotides in length will increase stability and selectivity of the hybrid, and thereby improving the specificity of particular hybrid molecules obtained. One can design nucleic acid molecules having gene-complementary stretches of more than about 25 and even more preferably more than about 50 nucleotides in length, or even longer where desired. Such fragments may be readily prepared by, for example, directly synthesizing the fragment by chemical means, by application of nucleic acid reproduction technology, such as the PCR™ technology with two priming oligonucleotides as described in U.S. Pat. No. 4,603,102 or by introducing selected sequences into recombinant vectors for recombinant production.

In certain embodiments, it will be advantageous to employ nucleic acid sequences of the present invention in combination with an appropriate means, such as a label, for detecting hybridization and therefore complementary sequences. A wide variety of appropriate indicator means are known in the art, including fluorescent, radioactive, enzymatic or other ligands, such as avidin/biotin, which are capable of giving a detectable signal. A fluorescent label or an enzyme tag, such as urease, alkaline phosphatase or peroxidase, instead of radioactive or other environmental undesirable reagents can also be used. In the case of enzyme tags, colorimetric indicator substrates are known which can be employed to provide a means visible to the human eye or spectrophotometrically, to identify specific hybridization with complementary nucleic acid-containing samples.

Hybridization reactions can be performed under conditions of different “stringency”. Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. Conditions that increase the stringency of a hybridization reaction are widely known and published in the art. See, Sambrook, et al. (1989) supra.

One can also utilize detect and quantify mRNA level or its expression using quantitative PCR or high throughput analysis such as Serial Analysis of Gene Expression (SAGE) as described in Velculescu, V. et al., Science (1995) 270:484-487. Briefly, the method comprises isolating multiple mRNAs from cell or tissue samples suspected of containing the transcript. Optionally, the gene transcripts can be converted to cDNA. A sampling of the gene transcripts are subjected to sequence-specific analysis and quantified. These gene transcript sequence abundances are compared against reference database sequence abundances including normal data sets for diseased and healthy patients. The patient has the disease(s) with which the patient's data set most closely correlates and for this application, includes the differential of the transcript.

The nucleotide probes of the present invention can also be used as primers for the amplification and detection of genes or gene transcripts. A primer useful for detecting differentially expressed mRNA is at least about 80% identical to the homologous region of comparable size of a gene or polynucleotide. For the purpose of this invention, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase.

General procedures for PCR are taught in MacPherson et al., PCR: A PRACTICAL APPROACH, (IRL Press at Oxford University Press (1991)). However, PCR conditions used for each application reaction are empirically determined. A number of parameters influence the success of a reaction. Among them are annealing temperature and time, extension time, Mg²⁺ ATP concentration, pH, and the relative concentration of primers, templates, and deoxyribonucleotides.

After amplification, the resulting DNA fragments can be detected by agarose gel electrophoresis followed by visualization with ethidium bromide staining and ultraviolet illumination. A specific amplification of differentially expressed genes of interest can be verified by demonstrating that the amplified DNA fragment has the predicted size, exhibits the predicated restriction digestion pattern, and/or hybridizes to the correct cloned DNA sequence.

Probes also can be attached to a solid support for use in high throughput screening assays using methods known in the art. International PCT Application No. WO 97/10365 and U.S. Pat. Nos. 5,405,783, 5,412,087 and 5,445,934, for example, disclose the construction of high density oligonucleotide chips which can contain one or more sequences. The chips can be synthesized on a derivatized glass surface using the methods disclosed in U.S. Pat. Nos. 5,405,783; 5,412,087 and 5,445,934. Photoprotected nucleoside phosphoramidites can be coupled to the glass surface, selectively deprotected by photolysis through a photolithographic mask, and reacted with a second protected nucleoside phosphoramidite. The coupling/deprotection process is repeated until the desired probe is complete.

The expression level of the gene is determined through exposure of a sample suspected of containing the polynucleotide to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See, U.S. Pat. Nos. 5,578,832 and 5,631,734. The obtained measurement is directly correlated with gene expression level.

The probes and high density oligonucleotide probe arrays also provide an effective means of monitoring expression of a multiplicity of genes, one of which includes the gene. Thus, the expression monitoring methods can be used in a wide variety of circumstances including detection of disease, identification of differential gene expression between samples isolated from the same patient over a time course, or screening for compositions that upregulate or downregulate the expression of the gene at one time, or alternatively, over a period of time.

Hybridized probe and sample nucleic acids can be detected by various methods known in the art. For example, the hybridized nucleic acids can be detected by detecting one or more labels attached to the sample nucleic acids. The labels can be incorporated by any of a number of means known to those of skill in the art. In one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., ³H, ¹²⁵I, ³⁵S, ¹⁴C, or ³²P) enzymes (e.g., horseradish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are known to those of skill in the art. Thus, for example, radiolabels may be detected using photographic film or scintillation counters, fluorescent markers can be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

Patent Publication WO 97/10365 describes methods for adding the label to the target (sample) nucleic acid(s) prior to or alternatively, after the hybridization. These are detectable labels that are directly attached to or incorporated into the target (sample) nucleic acid prior to hybridization. In contrast, “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids, see LABORATORY TECHNIQUES IN BIOCHEMISTRY AND MOLECULAR BIOLOGY, Vol. 24: Hybridization with Nucleic Acid Probes, P. Tijssen, ed. Elsevier, N.Y. (1993).

The nucleic acid sample also may be modified prior to hybridization to the high density probe array in order to reduce sample complexity thereby decreasing background signal and improving sensitivity of the measurement using the methods disclosed in International PCT Application No. WO 97/10365.

Results from the chip assay are typically analyzed using a computer software program. See, for example, EP 0 717 113 A2 and WO 95/20681. The hybridization data is read into the program, which calculates the expression level of the targeted gene(s). This figure is compared against existing data sets of gene expression levels for diseased and healthy individuals. A correlation between the obtained data and that of a set of diseased individuals indicates the onset of a disease in the subject patient.

### Diagnostic Methods for Detecting and Quantifying Protein or Polypeptides

In another aspect, the invention provides methods and compositions for diagnosing or monitoring cystic abnormalities such as those associated with ADPKD disease by detecting and/or quantifying protein or polypeptide expressed from a gene identified in Tables 2 through 5, infra, present in a sample. A variety of techniques are available in the art for protein analysis and include, but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays and PAGE-SDS.

In diagnosing disease characterized by a differential expression of the gene, one typically conducts a comparative analysis of the subject and appropriate controls. Preferably, a diagnostic test includes a control sample derived from a subject (hereinafter “positive control”), that exhibits the pathological or abnormal expression level of the gene. It is also useful to include a “negative control” that lacks the clinical characteristics of the pathological state and whose expression level of the gene is within a normal range. A positive correlation between the subject and the positive control with respect to the identified alterations indicates the presence of or a predisposition to disease. A lack of correlation between the subject and the negative control confirms the diagnosis.

One can also modify know immunoassays to detect and quantify expression. Determination of the gene product requires measuring the amount of immunospecific binding that occurs between an antibody reactive to the gene product. To detect and quantify the immunospecific binding, or signals generated during hybridization or amplification procedures, digital image analysis systems including but not limited to those that detect radioactivity of the probes or chemiluminescence can be employed.

### Methods to Identify Therapeutic Agents

The present invention also provides a screen for identifying leads and methods for reversing the pathological condition of the cells or tissues or selectively inhibiting growth or proliferation of the cells or tissues. In one aspect, the screen identifies lead compounds or biologics agents which are useful to treat cystic abnormalities or to treat or ameliorate the symptoms associated with ADPKD. The screens can be practiced in vitro or in vivo.

In one aspect, it is desirable to identify drug candidates capable of binding to soluble CTGF, or its receptor thereby inhibiting activation of the receptor. For some applications, the identification of drug candidates capable of blocking the protein from binding to its receptor will be desired. For some applications, the identification of a drug candidate capable of binding to the receptor may be used as a means to deliver a therapeutic or diagnostic agent. For other applications, the identification of drug candidates capable of mimicking the activity of the native CTGF will be desired. Thus, by manipulating the binding of a receptor:ligand complex, one may be able to promote or inhibit further development of cystic foci.

Test substances for screening can come from any source. They can be libraries of natural products, combinatorial chemical libraries, biological products made by recombinant libraries, etc. The source of the test substances is not critical to the invention. The present invention provides means for screening compounds and compositions which may previously have been overlooked in other screening schemes.

To practice the screen or assay in vitro, suitable cell cultures or tissue cultures are first provided. The cell can be a cultured cell or a genetically modified cell which differentially expresses the gene. Alternatively, the cells can be from a tissue biopsy. U.S. Pat. No. 5,789,189 provides a method of producing a culture of polycystic kidney cells which form cysts in vitro. The cells are cultured under conditions (temperature, growth or culture medium and gas (CO₂)) and for an appropriate amount of time to attain exponential proliferation without density dependent constraints. It also is desirable to maintain an additional separate cell culture; one which does not receive the agent being tested as a control.

As is apparent to one of skill in the art, suitable cells may be cultured in microtiter plates and several agents may be assayed at the same time by noting genotypic changes, phenotypic changes and/or cell death. In one aspect, the screen utilizes the compositions and methods of the MDCK cystic assay described infra.

When the agent is a composition other than a DNA or RNA nucleic acid molecule, the suitable conditions may be by directly added to the cell culture or added to culture medium for addition. As is apparent to those skilled in the art, an “effective” amount must be added which can be empirically determined.

The screen involves contacting the agent with a test cell differentially expressing the gene and then assaying the cell for the level of gene expression. In some aspects, it may be necessary to determine the level of gene expression prior to the assay. This provides a base line to compare expression after administration of the agent to the cell culture. In another embodiment, the test cell is a cultured cell from an established cell line that differentially expresses the CTGF gene. An agent is a possible therapeutic agent if gene expression is returned (reduced or increased) to a level that is present in a cell in a normal state.

In yet another aspect, the test cell or tissue sample is isolated from the subject to be treated and one or more potential agents are screened to determine the optimal therapeutic and/or course of treatment for that individual patient. For example, kidney or liver tissue is suitable for this assay.

For the purposes of this invention, an “agent” is intended to include, but not be limited to a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic compounds based on various core structures, and these are also included in the term “agent”. In addition, various natural sources can provide compounds for screening, such as plant or animal extracts, and the like. It should be understood, although not always explicitly stated that the agent is used alone or in combination with another agent, having the same or different biological activity as the agents identified by the inventive screen. The agents and methods also are intended to be combined with other therapies. They can be administered concurrently or sequentially.

Use of the screen in an animal such as a rat or mouse, the method provides a convenient animal model system which can be used prior to clinical testing of the therapeutic agent or alternatively, for lead optimization. In this system, a candidate agent is a potential drug, and may therefore be suitable for further development, if gene expression is returned to a normal level or if symptoms associated or correlated to the presence of cells containing differential expression of the CTGF gene are ameliorated, each as compared to untreated, animal having the pathological cells. It also can be useful to have a separate negative control group of cells or animals which are healthy and not treated, which provides a further basis for comparison.

### Diagnostic and Therapeutic Antibody Compositions

This invention also provides an antibody capable of specifically forming a complex with a protein or polypeptide of this invention, which are useful in the diagnostic and therapeutic methods of this invention. The term “antibody” includes polyclonal antibodies and monoclonal antibodies as well as derivatives thereof (described above). The antibodies include, but are not limited to mouse, rat, and rabbit or human antibodies. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. The antibodies are also useful to identify and purify therapeutic and/or diagnostic polypeptides.

Laboratory methods for producing polyclonal antibodies and monoclonal antibodies, as well as deducing their corresponding nucleic acid sequences, are known in the art, see Harlow and Lane (1988) supra and Sambrook et al. (1989) supra. The monoclonal antibodies of this invention can be biologically produced by introducing protein or a fragment thereof into an animal, e.g., a mouse or a rabbit. The antibody producing cells in the animal are isolated and fused with myeloma cells or hetero-myeloma cells to produce hybrid cells or hybridomas. Accordingly, the hybridoma cells producing the monoclonal antibodies of this invention also are provided.

For the purpose of illustration, the anti-CTGF antibody available under Catalog No. TP 143 (Torrey Pines Biolabs) or SC-14939 (Santa Cruz Biotechnology, Inc.) and known methods, one of skill in the art can produce and screen the hybridoma cells and antibodies of this invention for antibodies having the ability to bind CTGF proteins or polypeptides.

If a monoclonal antibody being tested binds with protein or polypeptide, then the antibody being tested and the antibodies provided by the hybridomas of this invention are equivalent. It also is possible to determine without undue experimentation, whether an antibody has the same specificity as the monoclonal antibody of this invention by determining whether the antibody being tested prevents a monoclonal antibody of this invention from binding the protein or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski, et al., Proc. Natl. Acad. Sci. USA (1985) 82:8653 or Spira, et al., J. Immunol. Methods (1984) 74:307.

This invention also provides biological active fragments of the polyclonal and monoclonal antibodies described above. These “antibody fragments” retain some ability to selectively bind with its antigen or immunogen. Such antibody fragments can include, but are not limited to Fab; Fab′; F(ab′)₂; Fv, and SCA.

A specific example of “a biologically active antibody fragment” is a CDR region of the antibody. Methods of making these fragments are known in the art, see for example, Harlow and Lane (1988) supra.

The antibodies of this invention also can be modified to create chimeric antibodies and humanized antibodies. Oi, et al., BioTechniques (1986) 4(3):214. Chimeric antibodies are those in which the various domains of the antibodies' heavy and light chains are coded for by DNA from more than one species.

The isolation of other hybridomas secreting monoclonal antibodies with the specificity of the monoclonal antibodies of the invention can also be accomplished by one of ordinary skill in the art by producing anti-idiotypic antibodies. Herlyn, et al., Science (1986) 232:100. An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

As used in this invention, the term “epitope” is meant to include any determinant having specific affinity for the monoclonal antibodies of the invention. Epitopic determinants usually consist of chemically active surface groupings of molecules such as amino acids or sugar side chains and usually have specific three dimensional structural characteristics, as well as specific charge characteristics.

The antibodies of this invention can be linked to a detectable agent or label. There are many different labels and methods of labeling known to those of ordinary skill in the art.

The coupling of antibodies to low molecular weight haptens can increase the sensitivity of the assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

The antibodies of the invention also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

Compositions containing the antibodies, fragments thereof or cell lines which produce the antibodies, are encompassed by this invention. When these compositions are to be used pharmaceutically, they can be combined with a pharmaceutically acceptable carrier.

The following experimental examples are intended to illustrate, not limit the invention.

### Experimental Methods

### Expression Analysis

A combination of two different expression profiling technologies were used to identify CTGF as a therapeutic target: SAGE (Velculescu, V. et al. (1995) supra) and microarray. Because SAGE can not be easily performed on a large number of samples, SAGE profiles of four immortalized normal and cystic cell lines were first generated and then, as a second step, a PKD cDNA custom microarray based on the SAGE findings and profile multiple ADPKD kidney samples was built. Combination of these high throughput genomic analyses not only give a snapshot of the gene expression profile but they also shed light on the disease molecular mechanism.

Comprehensive SAGE analysis (50,000-60,000 tags per library) was performed on liver and kidney epithelial cell lines generated from healthy or ADPKD affected donors in an effort to delineate functional groups and disease-specific pathways. See Tables 1A and 1B, infra. In conclusion, comprehensive gene expression profiles for normal and ADPKD phenotypes were generated. Multiple novel genes with potential roles in cystogenesis which fall into several functional pathways were identified (see Tables 2 through 5, infra). These pathways include proliferation, apoptosis, ECM remodeling and inflammation.

### High-Resolution 2D Gel Electrophoresis

High-resolution 2D gel electrophoresis was performed on cyst fluid isolated from a human patient as described Lopez (1999) Meth. Mol. Bio. 112:111-127 with the following minor modifications. Isoelectric focusing was performed using Immobiline dry strips (nonlinear pH range 3-10; Amersham Biosciences). The dry strips were rehydrated with protein sample (100 μg) by in-gel reswelling for 13 hours and electrophoresed (total 32 kVh). The second dimension was performed in 10% Duracryl polyacrylamide SDS gels run with the Investigator 2D electrophoresis system (Genomic Solutions, Ann Arbor, Mich., USA). Silver staining was performed as described in Rabilloud, “Methods in molecular biology: Proteom analysis protocols” (1999) 112:297-305. Gels were scanned using intensity calibrated Phoretix Power Scan Software v. 3.0 and analyzed by Phoretix Advanced software v.5.0. (Non linear Dynamics Ltd, Durham, N.C.). For identification of proteins using peptide mass fingerprinting, gels were stained with SYPRO Ruby protein gel stain (Molecular Probes, Eugene, Oreg., USA) according to the manufacturer specifications.

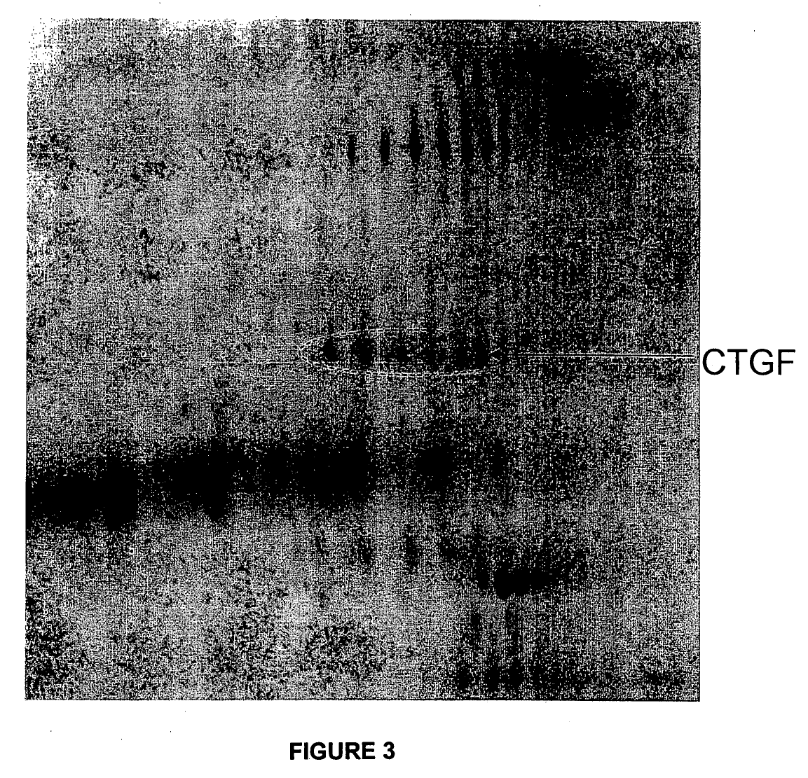

Results are shown in FIG. 3.

### Anti-CTGF Antibody Blocks Cyst Formation

MDCK cells are grown in complete MEM (minimal essential media) containing 10% heat inactivated fetal bovine serum, penicillin/streptomycin (Gibco, Rockville, Md.) until 80% confluent. MDCK cysts were grown as previously described in Pollack et al., Dev. Biol. (1998) 204:67-79. Briefly, MDCK cells were seeded into collagen matrix as follows: Day 1: To ensure they are in a logarithmic growth phase MDCK cells are trypsinized and expanded into 100 mm petri dishes (BD Biosciences). Day 2: mix on ice: 2.1 ml Type I rat tail collagen (BD Biosciences, Bedford, Mass.), 6.74 ml complete MEM, 0.98 ml 140 mM NaHCO₃ and 0.169 ml 142 mM NaOH, then transfer 50 μl into wells of 96 well plates (BD Biosciences) and incubate at 37° C. until solidified. The cells are trypsinized and plated on day 1 and resuspended with collagen mixture at a final concentration of 4×10⁴ cells/ml and distribute 50 μl/well. Incubate the cells at 37° C. for 30 minutes to allow collagen to solidify and overlay with 100 μl of complete MEM. On day 4, replace the media with complete MEM containing antibody. Media is refreshed every other day.

Two CTGF antibodies were tested in the MDCK in vitro cyst assay to assess their ability to block cyst formation. Commercial antibody preparations were resuspended as recommended by the manufacturer, dialysed against PBS to remove NaN3 and sterilized by 0.2 μm filtration before use. Rabbit anti-CTGF (Torrey Pines Biolabs, Inc., San Diego, Calif.), rabbit IgG control (Jackson ImmunoResearch Laboratories Inc., West Grove, Pa.) goat anti-CTGF antibody (Santa Cruz Biotechnology, Inc., Santa Cruz, Calif.) and goat IgG controls (Sigma/Aldrich) were used in 2 fold dilutions, starting from 25 μg/ml down to 0.02 μg/ml. Cysts were visualized with a Zeiss Axiovert 25 inverted microscope and Hamamatsu digital camera (Zeiss, Chester Va.) coupled to QED imaging software (QED Imaging Inc, Pittsburg, Pa.).

### CTGF Immunohistochemistry

Kidneys from jck (50 day old) or cpk (10 day old) mice were harvested from CO₂ asphyxiated animals (Jackson Laboratories, Bar Harbor, Me.). Sections (5 μm thick) were made from 4% paraformaldehyde-PBS fixed-paraffin embedded kidney and were incubated twice in 100% xylene for 5 min, twice in 100% ethanol for 5 min, twice in 95% ethanol for 5 min, twice in 80% ethanol for 5 min, twice in distilled water for 5 min and twice in phosphate buffered saline (PBS) for 5 min. Slides were blocked for 30 min in PBS containing 3% (w/vol) bovine serum albumin (PBS/BSA). Antigens were unmasked by incubating the section in trypsin solution (Sigma/Aldrich) for 30 min at room temperature, then washed with PBS 5 times for 5 min each. Sections were incubated in 20 μg/ml rabbit anti-CTGF (AbCam, Cambridge, Mass.) PBS/BSA for 2 h at room temperature followed by 5 PBS washes of 5 min each. Sections were incubated with anti-rabbit antibody (Sigma/Aldrich) at a 1:100 dilution (vol/vol) in PBS/BSA for 1 hr followed by 5 PBS washes of 5 min each. Slides were dipped in PBS containing 0.001% Evan's blue counterstain (Sigma/Aldrich) for 30 seconds followed by 5 PBS washes of 5 min each. Slides were mounted with mounting media (Vector Labs H1000) under glass coverslips. Slides were visualized with an Olympus IX70 microscope under UV illumination.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

TABLE 1A
Summary of SAGE libraries

| | Tags/ | Unique |
|---|---|---|
| Condition | library | Tags |
| NL | 53,176 | 18,965 |
| CL | 61,471 | 21,299 |
| NK | 51,923 | 19,378 |
| CK | 53,327 | 20,855 |

TABLE 1B

| CTGF expression | Normal Kidney | Cystic Kidney |
|---|---|---|
| Microarray (A.U.) | 5.028 | 7.434 |
| RT-Q PCR normalized to RPS | 0.601 ± 0.152 | 2.272 ± 0.647 |
| RT-Q PCR normalized to RPS | 0.413 ± 0.117 | 1.384 ± 0.273 |

A.U. arbitrary units

TABLE 2
Top 20 up- and down-regulated genes in CL.

| | | | | | | | |
|---|---|---|---|---|---|---|---|
| Tag Sequence | 11th | NL | CL | −NL/CL | Accession | Gene down-regulated (HUGO) | P value |

| | | | | | | | |
|---|---|---|---|---|---|---|---|
| AATCTGCGCC | | 22 | 0 | <−22 | M13755 | interferon-α inducible protein (G1P2) | 4.58E−07 |
| GCCCAGCTGG | A | 19 | 0 | <−19 | Z21507 | Eukaryotic translation elongation factor 1-δ (EEF1D) | 2.78E−06 |
| TCTGCACCTC | | 37 | 2 | −18.5 | X70940 | Eukaryotic translation elongation factor 1-α 2 (EEF1A2) | 1.26E−09 |
| TGCTGCCTGT | T | 18 | 1 | −18 | NM_004335 | Bone marrow stromal cell antigen 2 (BST2) | 2.37E−05 |
| GGGCCCCCTG | G | 15 | 0 | <−15 | NM_002101 | Glycophorin C (GYPC) | 3.12E−05 |
| GTGCAGGTCT | | 15 | 0 | <−15 | B1262403 | Hypothetical protein MGC4022 (R32184_3) | 3.12E−05 |
| AGGCAGACGG | | 14 | 1 | −14 | AL566171 | Eukaryotic translation elongation factor 1-α 2 (EEF1A2) | 2.66E−04 |
| CTTGGGAGGC | G | 14 | 1 | −14 | NM_032158 | KIAA0618 gene product (WBSCR20C) | 2.66E−04 |
| TGGGACGTGA | | 14 | 1 | −14 | NM_004445 | EphB6 (EPHB6) | 2.66E−04 |
| AGGAGGGAGG | C | 12 | 0 | <−12 | M77836 | Pyrroline-5-carboxylate reductase 1 (PYCR1) | 1.96E−04 |
| GCTCCCAGAC | | 12 | 1 | −12 | BC029755 | Synaptogyrin 2 (SYNGR2) | 8.98E−04 |
| GTGCTGATTC | T | 24 | 2 | −12 | L02870 | Collagen, type VII-α 1 (COL7A1) | 2.65E−06 |
| TGTGCGCGGG | | 12 | 1 | −12 | AF124432 | Intermediate filament-like MGC: 2625 (DKFZP58612223) | 8.98E−04 |
| TTCTCCCGCT | | 12 | 1 | −12 | NM_000308 | Protective protein for β-galactosidase (PPGB) | 8.98E−04 |
| ACTCGCTCTG | | 21 | 2 | −10.5 | NM_005560 | Laminin-α 5 (LAMA5) | 1.56E−05 |
| CCCTCAGCAC | C | 10 | 1 | −10 | BC004376 | Annexin A8 (ANXA8) | 3.06E−03 |
| CCGCTGCTTG | | 10 | 0 | <−10 | NM_006736 | DnaJ (Hsp40) homolog, subfamily B, member 2 (DNAJB2) | 6.74E−04 |
| CCTCTGGAGG | | 10 | 1 | −10 | BM969091 | P450 (cytochrome) oxidoreductase (POR) | 3.06E−03 |
| CTGACCCCCT | | 10 | 1 | −10 | NM_012200 | β-1,3-glucuronyltransferase 3 (B3GAT3) | 3.06E−03 |
| TTGACCCTGG | | 9 | 1 | −9 | NM_014338 | phosphatidylserine decarboxylase (PISD) | 5.68E−03 |

| | | | | | | | |
|---|---|---|---|---|---|---|---|
| Tag Sequence | 11th | NL | CL | CL/NL | Accession | Gene up-regulated (HUGO) | P value |

| | | | | | | | |
|---|---|---|---|---|---|---|---|
| TCAGGCCTGT | | 0 | 118 | >118 | X03655 | Granulocyte colony-stimulating factor (CSF3) | <1.0E−16 |
| TTGGTTTTTG | | 0 | 88 | >88 | U81234 | CXCL-6 chemokine (CXCL6) | <1.0E−16 |
| AGGTCCTAGC | | 0 | 76 | >76 | U62589 | Glutathione S-transferase P1c (GSTP1) | <1.0E−16 |
| ACCGCCGTGG | | 0 | 63 | >63 | M21186 | Cytocthrome b-245-α polypeptide (CYBA) | 1.54E−13 |
| GTTCACATTA | | 0 | 61 | >61 | X00497 | HLA-DR antigens associated invariant chain (CD74) | 3.73E−13 |
| GACCTGGAGC | C | 0 | 34 | >34 | NM_004417 | Dual specificity phosphatase 1 (DUSP1) | 5.84E−08 |
| TGCCCTCAGG | | 0 | 34 | >34 | AA169874 | Lipocalin 2 (LCN2) | 5.84E−08 |
| AGACCCCCAA | C | 0 | 32 | >32 | AI479224 | Myeloid/lymphoid or mixed-lineage leukemia3 (MLL3) | 1.43E−07 |
| TGCAGTCACT | G | 0 | 25 | >25 | X54925 | Matrix metalloproteinase 1 (MMP1) | 3.31E−06 |
| TGCCCTCAAA | | 0 | 23 | >23 | AA169874 | Lipocalin 2 (LCN2) | 8.17E−06 |
| GTCCCCACTG | | 1 | 23 | 23 | AI870124 | cDNA FLJ22487 fis, clone HRC10931 | 3.36E−05 |
| TGAGTCCCTG | | 1 | 18 | 18 | NM_004878 | Prostaglandin E synthase (PGES) | 3.25E−04 |
| GAAAAGTTTC | C | 2 | 35 | 17.5 | X78686 | CXCL-5 chemokine (CXCL5) | 5.77E−07 |
| TGGAAGCACT | T | 1 | 17 | 17 | M26383 | CXCL-8 chemokine (CXCL8) | 5.14E−04 |
| TGACTGGCAG | | 1 | 16 | 16 | BC001506 | CD59 antigen p18-20 (CD59) | 6.12E−04 |
| GGAAAAGTGG | T | 1 | 15 | 15 | V00496 | Serine (or cysteine) proteinase inhibitor, clade A1 | 1.29E−03 |
| | | | | | | (SERPINA1) | |
| TAGCAGCAAT | | 1 | 15 | 15 | NM_022154 | BCG-induced gene in monocytes (BICM103) | 1.29E−03 |
| ATAGCTGGGG | C | 1 | 15 | 15 | L11284 | Mitogen-activated protein kinase kinasa 1 (MAP2K1) | 1.29E−03 |
| TTGAATCCCC | | 0 | 14 | >14 | Z18538 | Protease inhibitor (PI3) | 5.01E−04 |
| TTGAAACTTT | A | U | 14 | >14 | J03561 | CXCL-1 chemokine (CXCL1) | 5.01E−04 |
| GACATCAAGT | C | 0 | 14 | >14 | Y00503 | Keratin 19 (KRT19) | 5.01E−04 |

TABLE 3
Top 20 up- and down-regulated genes in CK

| | | | | | | | |
|---|---|---|---|---|---|---|---|
| Tag Sequence | 11th | NK | CK | −NK/CK | Accession | Gene down-regulated (HUGO) | P value |

| | | | | | | | |
|---|---|---|---|---|---|---|---|
| GACCAGGCCC | | 33 | 1 | −33 | M12125 | Tropomyosin-1 (TPM1) | 2.60E−08 |
| CCCGAGGCAG | | 15 | 0 | <−15 | AB012664 | Stanniocalcin-2 (STC-2) | 8.67E−05 |
| TGCGGAGGCC | | 13 | 0 | 13 | AB001740 | Sjogren's syndrome/scleroderma autoantigen 1 (SSSCA1) | 3.82E−03 |
| GACTCGCTCC | | 13 | 0 | <−13 | BF718611 | cDNA for differentially expressed CO16 gene | 2.58E−04 |
| | | | | | | (HSJ001348) | |
| TGCAGCGCCT | | 11 | 1 | −11 | NM_003364 | Uridine phosphorylase (UP) | 3.35E−03 |
| ATGTGTGTTG | | 10 | 0 | <−10 | AF002697 | BCL2/adenovirus E1B 19 kDa interacting protein 3 | 1.35E−03 |
| | | | | | | (BNIP3) | |
| GCAATAAATG | G | 10 | 1 | −10 | D17530 | Drebrin (BN1) | 5.81E−03 |
| GTGGCGGGAG | | 9 | 1 | −9 | X64002 | General transcription factor IIF, polypeptide 1 | 1.00E−02 |
| | | | | | | (74 kD subunit) (GTF2F1) | |
| AAGGAAGCAA | T | 9 | 1 | −9 | Y64002 | Nucleolar protein 5A (56 kD with KKE/D repeat) (NOL5A) | 1.00E−02 |
| GGCGGCTGTG | | 8 | 0 | <−8 | AF014404 | Peroxisomal acyl-CoA thioesterase (PTE1) | 4.15E−03 |
| GGGCCCTTCC | T | 8 | 1 | −8 | AF055022 | Chromosome 20 open reading frame 188 (C20orf188) | 1.00E−02 |
| GGCAGCAATG | | 8 | 0 | <−8 | X03212 | Keratin 7 (KRT7) | 4.15E−03 |
| CGGCACATCC | | 8 | 1 | −8 | U26401 | Galactokinase (GALK1) | 1.00E−02 |
| TTCCCAAAGG | C | 8 | 1 | −8 | X91504 | ADP ribosylation factor related protein 1 (ARFRP1) | 1.00E−02 |
| AACCCTGCCC | C | 8 | 1 | −8 | U34683 | Glutathione synthetase (GSS) | 1.00E−02 |
| CCGCTGATCC | | 22 | 3 | −7.3 | S79639 | Exostoses (multiple) 1 (EXT1) | 1.10E−04 |
| GCCATAAAAT | | 7 | 0 | <−7 | X17042 | Proteoglycan 1, secetory granule (PRG1) | 7.33E−03 |
| GCAACGGGCC | | 14 | 2 | −7 | U91316 | Brain acyl-CoA hydrolase (BACH) | 2.26E−03 |
| GCCGGGTGGG | | 136 | 21 | −6.5 | D45131 | Basigin (OK blood group) (BSG) | <1.0E−16 |
| TGGACATCAT | | 5 | 0 | <−6 | NM_013334 | GDP-mannose pyrophosphorylase B (GMPPB) | 1.00E−02 |

| | | | | | | | |
|---|---|---|---|---|---|---|---|
| Tag Sequence | 11th | NK | CK | CK/NK | Accession | Gene up-regulated (HUGO) | P value |

| | | | | | | | |
|---|---|---|---|---|---|---|---|
| ATCTTGTTAC | | 1 | 56 | 56 | X02761 | Fibronectin 1 (FN1) | 6.68E−13 |
| GAAAAATACA | T | 1 | 49 | 49 | AL831902 | FLJ30315 fis, Clone BRACE2003539 (LOC162967) | 2.15E−11 |
| CCGCTATCCA | | 2 | 88 | 44 | | No match tag | <1.0E−16 |
| TTGGTTTTTG | | 0 | 29 | >29 | U81234 | CXCL-6 chemokine (CXCL6) | 1.07E.07 |
| GTTGTCTTTG | G | 2 | 51 | 25.5 | K02765 | Complement component C3 (C3) | 7.36E−12 |
| CTGAACCGGG | | 1 | 24 | 24 | | No match lag | 5.79E−06 |
| GCCCGGTGGG | C | 1 | 24 | 24 | | No match tag | 5.79E−06 |
| CCGGCCCTAC | | 3 | 60 | 20 | U21049 | Epithelial protein up regulated in carcinoma (DD96) | 1.47E−12 |
| TCACCTTAGG | T | 1 | 17 | 17 | NM_021999 | integral membrane protein 2B (ITM2B) | 4.73E−05 |
| AAGAGTTTTG | | 1 | 17 | 17 | X15414 | Aldo keto reductase family 1 member B1 (AKR1B1) | 2.03E−04 |
| TTGCTGCCAG | C | 1 | 16 | 16 | AW088077 | Molecule possessing ankyrin repeats induced by | 3.39E−04 |
| | | | | | | lipopolysaccharide (MAIL) | |
| CAATAAATGT | T | 0 | 16 | >16 | D23661 | Ribosomal protein L37 (RPL37) | 7.91E−05 |
| GCCACACCCA | C | 1 | 15 | 15 | AW264297 | C-type lectin, super family member 9 (CLECSF9) | 5.67E−04 |
| TGCCCTCAAA | | 0 | 15 | >15 | BE645920 | Lipocalin 2 (LCN2) | 1.32E−04 |
| TGCCCTCAGG | C | 2 | 28 | 14 | BE645920 | Lipocalin 2 (LCN2) | 2.94E−06 |
| ACACCTCTAA | A | 0 | 13 | >13 | BC001375 | Cytosolic non specific dipeptidase (CN2) | 3.74E−04 |
| GTGCGAAGGA | | 1 | 13 | 13 | | No match tag | 1.59E−03 |
| GTGCCGGAGG | | 0 | 12 | >12 | | No match tag | 6.30E−04 |
| TAAGTGTGGT | T | 0 | 11 | >11 | BU752045 | Claudin 1 (CLDN1) | 1.06E−03 |
| CCAGCTTCCT | | 1 | 12 | 12 | X58840 | Transcription factor 2, hepatic (TCF2) | 2.67E−03 |

TABLE 4

Up-regulated genes >5x common to CK and CL

| | | | | | |
|---|---|---|---|---|---|
| Tag | 11th | CL/NL | CK/NK | Accession # | Description (HUGO) |

| | | | | | |
|---|---|---|---|---|---|
| AGTATCTGGG | | 6 | 5 | AF006084 | Arp2/3 protein complex subunit 1B p41 |
| | | | | | (ARPC1B) |
| AAGTTGCTAT | | 10 | 5 | J03077 | β-glucosidase, prosaposin (PSAP) |
| TAGCAGCAAT | | 15 | >5 | NM_022154 | BCG-induced gene in monocytes (BICM103) |
| TATGAATGCT | | >6 | 10 | NM_004385 | Chondroitin sulfate proteoglycan (CSPG2) |
| GTCTTAAAGT | | 10 | >10 | BC016015 | Clone IMAGE 4711494 |
| AGATGAGATG | | 5 | 6 | AF001461 | Core promoter element binding protein |
| | | | | | (COPEB) |
| GAAAAGTTTC | C | 17.5 | 9 | X78686 | CXCL-5 chemokine (CXCL5) |
| TTGGTTTTTG | | >88 | >29 | U81234 | CXCL-6 chemokine (CXCL6) |
| CCGGCCCTAC | | 7.3 | 20 | U21049 | DD96 membrane associated (DD96) |
| CGCCCGTCGT | G | 8 | 5.5 | AL390147 | Hypothetical protein (DKZFp547D065) |
| GAAAAATACA | T | 7.4 | 49 | AL831902 | Hypothetical protein (LOC162967) |
| ACAGAAGGGA | G | 6 | >7 | U28252 | β1 integrin (ITGB1) |
| TGCCCTCAAA | A | >23 | >15 | BE645920 | Lipocalin-2 (LCN2) |
| TGCCCTCAGG | | >34 | 14 | BE645920 | Lipocalin-2 (LCN2) |
| GGGATTAAAG | | 5 | 8 | M28882 | Melanoma cell adhesion molecule (MCAM) |
| TTCTATTTCA | | 7 | 6 | M69066 | Moesin (MSN) |
| CCTGAGGAAT | | >5 | >5 | NM_031419 | Molecule possessing ankyrin repeats |
| | | | | | induced by lipopolysaccharide (MAIL) |
| TTGCTGCCAG | C | 12 | 16 | NM_031419 | Molecule processing ankyrin repeats |
| | | | | | induced by lipopolysaccharide (MAIL) |
| GTCGAAGGAC | | >6 | >25 | | No match tag |
| GTGCCGGAGG | | 5.5 | >12 | | No match tag |
| GTGCGAAGGA | | >7 | 13 | | No match tag |
| TCGCTGCTTT | | >381 | 6 | | No match tag |
| TGGTGTTAAG | | 11 | 6 | X69150 | Ribosomal protein S18 (RPS18) |
| CCTATGTAAG | | 8 | >6 | Z23064 | RNA binding motif protein X chromosome |
| | | | | | (RBMX) |
| GGAAAAGTGG | T | 15 | 10.5 | X01683 | Serine (or cysteine) proteinase inhibi- |
| | | | | | tor, clade A1 (SERPINA1) |
| GTGCGGAGGA | C | 5.1 | 9.3 | M10906 | Serum amyloid A1 (SAA1) |
| TTGGGGGTTT | | 6.3 | >7 | NM_003599 | Suppressor of Ty 3 homolog (SUPT3H) |
| TCTGCAAATT | | >5 | >5 | NM_032525 | Tubulin β 5 (TUBB-5) |

TABLE 5-A

Functional groups of genes up-regulated in CL.

| | | | | | | |
|---|---|---|---|---|---|---|
| | | | | | Acces- | |
| Tag | NL | CL | NK | CK | sion | Gene (HUGO) |

1-Growth factors, chemokines and inflammatory
response related

| | | | | | | |
|---|---|---|---|---|---|---|
| TCAGGCCTGT | 0 | 118 | 0 | 0 | X03655 | Granulocyte colo- |
| | | | | | | ny-stimulating |
| | | | | | | factor (CSF3) |
| GAAAAGTTTC | 2 | 35 | 1 | 9 | X78686 | CXCL-5 chemokine |
| | | | | | | (CXCL5) |
| TTGAAACTTT | 0 | 14 | 0 | 3 | J03561 | CXCL-1 chemokine |
| | | | | | | (CXCL1) |
| TGGAAGCACT | 1 | 17 | 1 | 3 | X78686 | CXCL-8 chemokine |
| | | | | | | (CXCL8) |

2-Receptors and cell surface antigens

| | | | | | | |
|---|---|---|---|---|---|---|
| GGAGGTAGGG | 1 | 11 | 5 | 5 | U40271 | Transmembrane re- |
| | | | | | | ceptor precursor |
| | | | | | | (PTK7) |
| CTGTGAGACC | 0 | 8 | 1 | 0 | U12255 | Fc fragment of IgG |
| | | | | | | receptor, trans- |
| | | | | | | porter-α (FCGRT) |
| TGGTCCAGCG | 1 | 7 | 0 | 0 | M86511 | Monocyte antigen |
| | | | | | | CD14 (CD14) |
| GTTCACATTA | 0 | 61 | 0 | 2 | X00497 | HLA-DR antigens |
| | | | | | | associated invari- |
| | | | | | | ant chain (CD74) |
| TGACTGGCAG | 1 | 16 | 6 | 10 | BC001506 | CD59 antigen p18- |
| | | | | | | 20 (CD59) |
| GCAGTTCTGA | 0 | 6 | 0 | 0 | X00700 | Fragment for class |
| | | | | | | II histocompati- |
| | | | | | | bility antigen |
| | | | | | | (HLA-DR) |
| ACAGAAGGGA | 0 | 6 | 2 | 14 | U28252 | β1 integrin |
| | | | | | | (ITGB1) |

3-Transcription factors and signal transduction
modulators

| | | | | | | |
|---|---|---|---|---|---|---|
| ATAGCTGGGG | 1 | 15 | 0 | 1 | L11284 | Mitogen-activated |
| | | | | | | protein kinase |
| | | | | | | kinase 1 (MAP2K1) |
| ACTGAGGAAA | 0 | 6 | 3 | 6 | M31159 | IGFBP 3 |
| ATCAAATGCA | 1 | 5 | 0 | 3 | K02276 | c-Myc (MYC) |
| GGAGGTAGGG | 1 | 11 | 5 | 5 | U33635 | Protein tyrosine |
| | | | | | | kinase 7 (PTK7) |
| GGATGCAAGG | 1 | 5 | 0 | 0 | U07349 | Mitogen-activated |
| | | | | | | protein kinase |
| | | | | | | kinase kinase |
| | | | | | | kinase 2 (MAP4K2) |
| GACCTCCTGC | 1 | 5 | 2 | 4 | L32976 | Mitogen-activated |
| | | | | | | protein kinase |
| | | | | | | kinase kinase 11 |
| | | | | | | (MAP3K11) |

5-Cytoskeleton

| | | | | | | |
|---|---|---|---|---|---|---|
| TTCTATTTCA | 1 | 7 | 1 | 6 | M69066 | Moesin (MSN) |
| AGTATCTGGG | 1 | 6 | 1 | 5 | AF006084 | Arp2/3 protein |
| | | | | | | complex subunit 1B |
| | | | | | | p41 (ARPC1B) |
| CTGGCGCGAG | 0 | 13 | 0 | 1 | X69549 | Rho-GDP dissocia- |
| | | | | | | tion inhibitor-β |
| | | | | | | (GDI)(ARHGDIB) |

6-Extra-cellular matrix

| | | | | | | |
|---|---|---|---|---|---|---|
| ACAGAGCACA | 0 | 11 | 0 | 0 | X91171 | laminin α 4 |
| | | | | | | (LAMA4) |
| 7-Proteases | | | | | | |
| TGCAGTCACT | 0 | 25 | 0 | 0 | M13509 | Matrix metalo pro- |
| | | | | | | tease 1 (MMP1) |
| GGAAAAGTGG | 1 | 15 | 2 | 21 | V00496 | Serine (or cy- |
| | | | | | | steine) proteinase |
| | | | | | | inhibitor, clade |
| | | | | | | A1 (SERPINA1) |
| TTTCCCTCAA | 3 | 16 | 0 | 2 | D87258 | Serine protease 11 |
| | | | | | | with IGF binding |
| | | | | | | (PRSS11) |
| TTGATGCCCG | 0 | 5 | 0 | 3 | M93056 | Serine (or cy- |
| | | | | | | steine) proteinase |
| | | | | | | inhibitor, clade |
| | | | | | | B1 (SERPINB1) |

8-Ion channels and transporters

| | | | | | | |
|---|---|---|---|---|---|---|
| TATGACTTAA | 1 | 7 | 2 | 2 | AF031815 | Potassium inter- |
| | | | | | | mediate/small con- |
| | | | | | | ductance calcium- |
| | | | | | | activated channel, |
| | | | | | | subfamily N, mem- |
| | | | | | | ber 3 (KCNN3) |

9-Miscellaneous

| | | | | | | |
|---|---|---|---|---|---|---|
| AGGTTTCCTC | 1 | 8 | 4 | 2 | D67025 | Proteasome 26S |
| | | | | | | subunit, non- |
| | | | | | | ATPase, 3 (PSMD3) |
| ATGGGATGGC | 1 | 5 | 0 | 0 | J02761 | Surfactant, pul- |
| | | | | | | monary-associated |
| | | | | | | protein B (SFTPB) |
| CCCAACGCGC | 0 | 10 | 0 | 0 | V00493 | Hemoglobin-α 2 |
| | | | | | | (HBA2) |
| CCCGAGGCAG | 1 | 9 | 15 | 0 | AF055460 | Stanniocalcin 2 |
| | | | | | | (STC2) |
| CTTTGAGTCC | 0 | 9 | 0 | 0 | U01101 | Uteroglobin, fami- |
| | | | | | | ly 1A, member 1 |
| | | | | | | (SCGB1A1) |
| GATGCGAGGA | 2 | 12 | 1 | 0 | U38276 | Semaphorin 3F |
| | | | | | | (SEMA3F) |
| GCAAGAAAGT | 0 | 5 | 0 | 0 | M25113 | Hemoglobin-β (HBB) |
| GCAGGCCAAG | 4 | 24 | 1 | 0 | U57092 | B-factor, proper- |
| | | | | | | din (BF) |
| GCCTTCCAAT | 4 | 21 | 4 | 11 | X52104 | RNA helicase, 68 |
| | | | | | | kDa (DDX5) |
| GGGATTAAAG | 1 | 5 | 1 | 8 | M28882 | Melanoma cell ad- |
| | | | | | | hesion molecule |
| | | | | | | (MCAM) |
| GTAATGACAG | 1 | 6 | 1 | 0 | U25997 | Stanniocalcin 1 |
| | | | | | | (STC1) |
| GTCTGGGGGA | 0 | 7 | 3 | 8 | U67963 | Monoglyceride li- |
| | | | | | | pase (MGLL) |
| GTGCGGAGGA | 61 | 312 | 45 | 418 | M23698 | Serum amyloid A1 |
| | | | | | | (SAA1) |
| GTGGTGGACA | 1 | 5 | 12 | 3 | U68041 | Breast cancer 1, |
| | | | | | | early onset |
| | | | | | | (BRCA1) |
| GTGTCTCGCA | 2 | 12 | 3 | 7 | L19605 | Annexin A11 |
| | | | | | | (ANXA11) |
| TGGAAAGCTT | 1 | 15 | 4 | 3 | M64497 | Nuclear receptor |
| | | | | | | subfamily 2F2 |
| | | | | | | (NR2F2) |
| TGGCTTGCTC | 2 | 15 | 3 | 6 | AF069250 | Okadaic acid- |
| | | | | | | inducible phospho- |
| | | | | | | protein (OA48-18) |
| TTGAATCCCC | 0 | 14 | 0 | 1 | Z18538 | Protease inhibitor |
| | | | | | | 3, skin-derived |
| | | | | | | (PI3) |

TABLE 5-B

Functional groups of genes up-regulated in CK.

| | | | | | | |
|---|---|---|---|---|---|---|
| | | | | | Acces- | |
| Tag | NL | CL | NK | CK | sion | Gene (HUGO) |

1-Growth factors, chemokines and inflammatory
response related

| | | | | | | |
|---|---|---|---|---|---|---|
| GACGGCGCAG | 13 | 3 | 0 | 6 | M63193 | Endothelial cell |
| | | | | | | growth factor 1 |
| | | | | | | (ECGF1) |
| TTTGCACCTT | 2 | 7 | 10 | 2 | X78947 | Connective tissue |
| | | | | | | growth factor |
| | | | | | | (CTGF) |
| GAAAAGTTTC | 2 | 35 | 1 | 9 | X78886 | CXCL-5 chemokine |
| | | | | | | (CXCL5) |
| TTGAAACTTT | 0 | 14 | 0 | 3 | J03561 | CXCL-1 chemokine |
| | | | | | | (CXCL1) |

2-Receptors and cell surface antigens

| | | | | | | |
|---|---|---|---|---|---|---|
| AAGATTGGGG | 3 | 5 | 1 | 11 | U40373 | Cell surface gly- |
| | | | | | | coprotein CD44 |
| | | | | | | (CD44) |
| GTACGGAGAT | 0 | 0 | 0 | 9 | M30257 | Vascular cell ad- |
| | | | | | | hesion molecule 1 |
| | | | | | | (VCAM1) |
| TTCAGGAGGG | 2 | 9 | 1 | 6 | M17661 | T cell receptor-α |
| | | | | | | locus (TRA@) |
| TCGAAGAACC | 3 | 7 | 1 | 6 | M59907 | CD 63 Melanoma 1 |
| | | | | | | antigen (CD63) |
| AAAACTGAGA | 6 | 1 | 0 | 5 | Z50022 | Pituitary tumor- |
| | | | | | | transforming 1 in- |
| | | | | | | teracting protein |
| | | | | | | (PTTG1IP) |
| ACAGAAGGGA | 0 | 6 | 2 | 14 | U28252 | β1 integrin |
| | | | | | | (ITGB1) |
| CCAGGCTGCG | 9 | 12 | 2 | 10 | M35011 | β5 integrin |
| | | | | | | (ITGB5) |
| GTACTGTAGC | 5 | 22 | 4 | 20 | M59911 | α3 integrin |
| | | | | | | (ITGA3) |

3-Transcription factors and signal transduction
modulators

| | | | | | | |
|---|---|---|---|---|---|---|
| ATCAAATGCA | 1 | 5 | 0 | 3 | K02276 | c-Myc (MYC) |
| GGAGGGATCA | 10 | 8 | 1 | 8 | U40282 | Integrin linked |
| | | | | | | kinase (ILK) |
| ATGGCCATAG | 6 | 2 | 1 | 6 | X99325 | Serine/threonine |
| | | | | | | kinase 25 (STK25) |
| CAGCGCCACC | 5 | 7 | 1 | 5 | AF035625 | Serine/threonine |
| | | | | | | kinase 11 (STK11) |
| 4-Apoptosis | | | | | | |
| ACCATCCTGC | 3 | 11 | 1 | 5 | AF039067 | anti-death protein |
| | | | | | | IEX-1L(IER3) |
| AAAGTCTAGA | 0 | 3 | 0 | 5 | M73554 | bcl-1 (CCND1) |

5-Cytoskeleton

| | | | | | | |
|---|---|---|---|---|---|---|
| TTCCACTAAC | 9 | 14 | 0 | 5 | U53204 | Plectin 1 inter- |
| | | | | | | mediate filament |
| | | | | | | (PLEC1) |
| TTCTATTTCA | 1 | 7 | 1 | 6 | M69066 | Moesin (MSN) |
| AGTATCTGGG | 1 | 6 | 1 | 5 | AF006084 | Arp2/3 protein |
| | | | | | | complex subunit 1B |
| | | | | | | p41 (ARPC1B) |

6-Extra-cellular matrix

| | | | | | | |
|---|---|---|---|---|---|---|
| ATCTTGTTAC | 3 | 11 | 1 | 56 | W47550 | Fibronectin 1 |
| | | | | | | (FN1) |
| 7-Proteases | | | | | | |
| GGAAAAGTGG | 1 | 15 | 2 | 21 | V00496 | Serine (or Cy- |
| | | | | | | steine) proteinase |
| | | | | | | inhibitor, clade |
| | | | | | | A1 (SERPINA1) |
| GCACCTGTCG | 19 | 22 | 0 | 12 | X13276 | Aminopeptidase N, |
| | | | | | | CD13 (ANPEP) |
| GCAAAAAAAA | 11 | 11 | 1 | 10 | AF053944 | Aortic carboxy |
| | | | | | | peptidase like |
| | | | | | | (AEBP1) |

8-Ion channels and transporters

| | | | | | | |
|---|---|---|---|---|---|---|
| GATCCTGGAT | 0 | 0 | 1 | 5 | Y17975 | ATPase, H+ trans- |
| | | | | | | porting, lysosomal |
| | | | | | | interacting pro- |
| | | | | | | tein 2 (ATP6/P2) |
| TTCACTGCCG | 6 | 3 | 1 | 9 | D49400 | ATPase, H+ trans- |
| | | | | | | porting, lysosomal |
| | | | | | | 14 kDa, V1 subunit |
| | | | | | | F (ATP6V1F) |
| ACAAACCCCC | 2 | 6 | 0 | 2 | W37827 | ATPase, Na+/K+ |
| | | | | | | transporting β 1 |
| | | | | | | polypeptide |
| | | | | | | (ATP1B1) |
| CACAGTCAAA | 1 | 5 | 0 | 1 | R42029 | β-3 subunit vol- |
| | | | | | | tage-dependent |
| | | | | | | calcium channel |
| | | | | | | (CACNB3) |

9-Miscellaneous

| | | | | | | |
|---|---|---|---|---|---|---|
| AAGCAGGAGG | 0 | 3 | 1 | 8 | U68019 | MAD mothers a- |
| | | | | | | gainst decapentap- |
| | | | | | | legic homolog 3 |
| | | | | | | (MADH3) |
| AATGCTTGAT | 1 | 3 | 1 | 6 | U35143 | Retinoblastoma |
| | | | | | | binding protein 7 |
| | | | | | | (RBBP7) |
| ACAAATCCTT | 4 | 13 | 1 | 6 | M34539 | FK506 binding pro- |
| | | | | | | tein 1A, 12 kDa |
| | | | | | | (FKBP1A) |
| ACTCAGCCCG | 10 | 16 | 1 | 8 | M92357 | Tumor necrosis |
| | | | | | | factor-α-induced |
| | | | | | | protein 2 |
| | | | | | | (TNFAIP2) |
| CACACCCCTG | 4 | 6 | 0 | 6 | Y12711 | Progesterone re- |
| | | | | | | ceptor membrane |
| | | | | | | component 1 |
| | | | | | | (PGRMC1) |
| CCAGGGGAGA | 2 | 3 | 0 | 6 | X67325 | Interferon-α- |
| | | | | | | inducible protein |
| | | | | | | 27 (IFI27) |
| CGACCCCACG | 13 | 2 | 0 | 5 | K00396 | Apolipoprotein E |
| | | | | | | (APOE) |
| GCTGCCCGGC | 6 | 9 | 1 | 7 | AF069733 | Transcriptional |
| | | | | | | adaptor 3 |
| | | | | | | (TADA3L) |
| TAAAAATGTT | 1 | 1 | 0 | 8 | M14083 | Serine (or cy- |
| | | | | | | steine) proteinase |
| | | | | | | inhibitor, clade |
| | | | | | | E1 (SERPINE1) |

1. A method for inhibiting cystic disorders or abnormalities in a suitable tissue by contacting the tissue with an effective amount of an agent that modulates the biological activity of a gene or polynucleotide identified in Tables 2 through 5, thereby inhibiting cystic abnormalities.

2. The method of claim 1, wherein the suitable tissue is selected from the group consisting of tubular kidney tissue, hepatic tissue and pancreatic tissue.

3. The method of claim 2, wherein the suitable tissue is isolated from a subject suffering from ADPKD.

4. The method of claim 1, wherein the modulating agent is a polynucleotide that modulates the activity or expression of a polynucleotide or gene identified in Tables 2 through 5.

5. The method of claim 4, wherein the agent is an antibody or ligand that specifically binds to the expression product of the gene or polynucleotide.

6. The method of claim 4, wherein the agent is selected from the group consisting of an antisense polynucleotide, a ribozyme and a multivalent RNA aptamer.

7. The method of claim 5, wherein the agent is selected from the group consisting of an antibody, an antibody derivative, and an antibody variant.

8. The method of claim 5, wherein the agent is a polyclonal antibody or a monoclonal antibody.

9. The method of claim 7, wherein the agent is selected from the group consisting of an antibody fragment, a humanized antibody and a chimeric antibody.

10. The method of claim 1, wherein the agent is a small molecule that modifies, blocks or augments post-translational modification the expression product of the gene or polynucleotide.

11. The method of claim 1, wherein the agent is a small molecule that modulates the activation of a precursor of the expression product of the polynucleotide or gene.

12. A method for inhibiting the formation of polycystic lesions in a subject, comprising delivering to the subject an effective amount of an agent that modulates the biological activity of a gene identified in Tables 2 through 5, thereby inhibiting the formation of polycystic lesions.

13. The method of claim 12, wherein the modulating agent is a polynucleotide that inhibits or augments the activity or expression of a CTGF polynucleotide.

14. The method of claim 12, wherein the agent is an antibody or ligand that specifically binds to the expression product of the gene or the polynucleotide.

15. The method of claim 13, wherein the agent is selected from the group consisting of an antisense polynucleotide, a ribozyme and a multivalent RNA aptamer.

16. The method of claim 14, wherein the agent is an antibody selected from the group consisting of a monoclonal antibody, an antibody derivative and an antibody variant.

17. The method of claim 14, wherein the agent is a polyclonal antibody or a monoclonal antibody.

18. The method of claim 17, wherein the agent is selected from the group consisting of an antibody fragment, a humanized antibody and a chimeric antibody.

19. The method of claim 12, wherein the agent is a small molecule that modifies, blocks, or augments post-translational modification of a polynucleotide or gene identified in Tables 2 through 5.

20. The method of claim 12, wherein the agent is a molecule that modifies the activation of a precursor of the expression product of the polynucleotide or gene identified in Tables 2 through 5.

21. A method for preventing or treating Autosomal Dominant Polycystic Kidney Disease (ADPKD) in a suitable subject, comprising delivering an effective amount of an isolated molecule that modulates polycystic biological activity of a gene or its expression product identified in Tables 2 through 5, to a subject in need thereof.

22. The method of claim 21, wherein the isolated molecule is a polynucleotide that modulates the activity or expression of a CTGF polynucleotide.

23. The method of claim 21, wherein the molecule is an antibody that specifically binds to the expression product of the gene or the polynucleotide.

24. The method of claim 22, wherein the molecule is selected from the group consisting of an antisense polynucleotide, a small molecule, a ribozyme, a multivalent RNA aptamer.

25. The method of claim 22, wherein the molecule is an antibody selected from the group consisting of an antibody, an antibody derivative and an antibody variant.

26. The method of claim 22, wherein the agent is a polyclonal antibody or a monoclonal antibody.

27. The method of claim 26, wherein the agent is selected from the group consisting of an antibody fragment, a humanized antibody and a chimeric antibody.

28. The method of claim 22, wherein the molecule is a small molecule that modifies, inhibits or augments post-translational modification of an expression product of a gene or a polynucleotide or gene identified in Tables 2 through 5.

29. The method of claim 22, wherein the molecule is a molecule that modifies the activation of a precursor of the expression product of a polynucleotide or gene identified in Tables 2 through 5.


SEQUENCE LISTING (193 sequences)

SEQ ID NO: 1 - DNA, 2312 bp, Homo sapiens
tccagtgacggagccgcccggccgacagccccgagacgacagcccggcgcgtcccggtccccacctccgaccaccgccagcgctccaggccccgcgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcttccccagacactggtttgaagaatgttaagacttgacagtggaactacattagtacacagcaccagaatgtatattaaggtgtggctttaggagcagtgggagggtaccagcagaaaggttagtatcatcagatagctcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttcctgtaacaagccagattttttaaaatttatattgtaaatattgtgtgtgtgtgtgtgtgtgtatatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctctcagatagaatgacagtccgtcaaaacagattgtttgcaaaggggaggcatcagtgtccttggcaggctgatttctaggtaggaaatgtggtagctcacgctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtatattttttctaaaaaaaaaaaaaaacgacagcaacggaattc

SEQ ID NO: 2 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 3 - DNA, 10 bp, Homo sapiens
aatctgcgcc

SEQ ID NO: 4 - protein, 10 aa, Homo sapiens
GCCCAGCTGG

SEQ ID NO: 5 - DNA, 10 bp, Homo sapiens
tctgcacctc

SEQ ID NO: 6 - DNA, 10 bp, Homo sapiens
tgctgcctgt

SEQ ID NO: 7 - DNA, 10 bp, Homo sapiens
gggccccctg

SEQ ID NO: 8 - DNA, 10 bp, Homo sapiens
gtgcaggtct

SEQ ID NO: 9 - DNA, 10 bp, Homo sapiens
aggcagacgg

SEQ ID NO: 10 - DNA, 10 bp, Homo sapiens
cttgggaggc

SEQ ID NO: 11 - DNA, 10 bp, Homo sapiens
tgggacgtga

SEQ ID NO: 12 - DNA, 10 bp, Homo sapiens
aggagggagg

SEQ ID NO: 13 - DNA, 10 bp, Homo sapiens
gctcccagac

SEQ ID NO: 14 - DNA, 10 bp, Homo sapiens
gtgctgattc

SEQ ID NO: 15 - DNA, 10 bp, Homo sapiens
tgtgcgcggg

SEQ ID NO: 16 - DNA, 10 bp, Homo sapiens
ttctcccgct

SEQ ID NO: 17 - DNA, 10 bp, Homo sapiens
actcgctctg

SEQ ID NO: 18 - DNA, 10 bp, Homo sapiens
ccctcagcac

SEQ ID NO: 19 - DNA, 10 bp, Homo sapiens
ccgctgcttg

SEQ ID NO: 20 - DNA, 10 bp, Homo sapiens
cctctggagg

SEQ ID NO: 21 - DNA, 10 bp, Homo sapiens
ctgaccccct

SEQ ID NO: 22 - DNA, 10 bp, Homo sapiens
ttgaccctgg

SEQ ID NO: 23 - DNA, 10 bp, Homo sapiens
tcaggcctgt

SEQ ID NO: 24 - DNA, 10 bp, Homo sapiens
ttggtttttg

SEQ ID NO: 25 - DNA, 10 bp, Homo sapiens
aggtcctagc

SEQ ID NO: 26 - DNA, 10 bp, Homo sapiens
accgccgtgg

SEQ ID NO: 27 - DNA, 10 bp, Homo sapiens
gttcacatta

SEQ ID NO: 28 - DNA, 10 bp, Homo sapiens
gacctggagc

SEQ ID NO: 29 - DNA, 10 bp, Homo sapiens
tgccctcagg

SEQ ID NO: 30 - DNA, 10 bp, Homo sapiens
agacccccaa

SEQ ID NO: 31 - DNA, 10 bp, Homo sapiens
tgcagtcact

SEQ ID NO: 32 - DNA, 10 bp, Homo sapiens
tgccctcaaa

SEQ ID NO: 33 - DNA, 10 bp, Homo sapiens
gtccccactg

SEQ ID NO: 34 - DNA, 10 bp, Homo sapiens
tgagtccctg

SEQ ID NO: 35 - DNA, 10 bp, Homo sapiens
gaaaagtttc

SEQ ID NO: 36 - DNA, 10 bp, Homo sapiens
tggaagcact

SEQ ID NO: 37 - DNA, 10 bp, Homo sapiens
tgactggcag

SEQ ID NO: 38 - DNA, 10 bp, Homo sapiens
ggaaaagtgg

SEQ ID NO: 39 - DNA, 10 bp, Homo sapiens
tagcagcaat

SEQ ID NO: 40 - DNA, 10 bp, Homo sapiens
atagctgggg

SEQ ID NO: 41 - DNA, 10 bp, Homo sapiens
ttgaatcccc

SEQ ID NO: 42 - DNA, 10 bp, Homo sapiens
ttgaaacttt

SEQ ID NO: 43 - DNA, 10 bp, Homo sapiens
gacatcaagt

SEQ ID NO: 44 - DNA, 10 bp, Homo sapiens
gaccaggccc

SEQ ID NO: 45 - DNA, 10 bp, Homo sapiens
cccgaggcag

SEQ ID NO: 46 - DNA, 10 bp, Homo sapiens
tgcggaggcc

SEQ ID NO: 47 - DNA, 10 bp, Homo sapiens
gactcgctcc

SEQ ID NO: 48 - DNA, 10 bp, Homo sapiens
tgcagcgcct

SEQ ID NO: 49 - DNA, 10 bp, Homo sapiens
atgtgtgttg

SEQ ID NO: 50 - DNA, 10 bp, Homo sapiens
gcaataaatg

SEQ ID NO: 51 - DNA, 10 bp, Homo sapiens
gtggcgggag

SEQ ID NO: 52 - DNA, 10 bp, Homo sapiens
aaggaagcaa

SEQ ID NO: 53 - DNA, 10 bp, Homo sapiens
ggcggctgtg

SEQ ID NO: 54 - DNA, 10 bp, Homo sapiens
gggcccttcc

SEQ ID NO: 55 - DNA, 10 bp, Homo sapiens
ggcagcaatg

SEQ ID NO: 56 - DNA, 10 bp, Homo sapiens
cggcacatcc

SEQ ID NO: 57 - DNA, 10 bp, Homo sapiens
ttcccaaagg

SEQ ID NO: 58 - DNA, 10 bp, Homo sapiens
aaccctgccc

SEQ ID NO: 59 - DNA, 10 bp, Homo sapiens
ccgctgatcc

SEQ ID NO: 60 - DNA, 10 bp, Homo sapiens
gccataaaat

SEQ ID NO: 61 - DNA, 10 bp, Homo sapiens
gcaacgggcc

SEQ ID NO: 62 - DNA, 10 bp, Homo sapiens
gccgggtggg

SEQ ID NO: 63 - DNA, 10 bp, Homo sapiens
tggacatcat

SEQ ID NO: 64 - DNA, 10 bp, Homo sapiens
atcttgttac

SEQ ID NO: 65 - DNA, 10 bp, Homo sapiens
gaaaaataca

SEQ ID NO: 66 - DNA, 10 bp, Homo sapiens
ccgctatcca

SEQ ID NO: 67 - DNA, 10 bp, Homo sapiens
ttggtttttg

SEQ ID NO: 68 - DNA, 10 bp, Homo sapiens
gttgtctttg

SEQ ID NO: 69 - DNA, 10 bp, Homo sapiens
ctgaaccggg

SEQ ID NO: 70 - DNA, 10 bp, Homo sapiens
gcccggtggg

SEQ ID NO: 71 - DNA, 10 bp, Homo sapiens
ccggccctac

SEQ ID NO: 72 - DNA, 10 bp, Homo sapiens
tcaccttagg

SEQ ID NO: 73 - DNA, 10 bp, Homo sapiens
aagagttttg

SEQ ID NO: 74 - DNA, 10 bp, Homo sapiens
ttgctgccag

SEQ ID NO: 75 - DNA, 10 bp, Homo sapiens
caataaatgt

SEQ ID NO: 76 - DNA, 10 bp, Homo sapiens
gccacaccca

SEQ ID NO: 77 - DNA, 10 bp, Homo sapiens
tgccctcaaa

SEQ ID NO: 78 - DNA, 10 bp, Homo sapiens
tgccctcagg

SEQ ID NO: 79 - DNA, 10 bp, Homo sapiens
acacctctaa

SEQ ID NO: 80 - DNA, 10 bp, Homo sapiens
gtgcgaagga

SEQ ID NO: 81 - DNA, 10 bp, Homo sapiens
gtgccggagg

SEQ ID NO: 82 - DNA, 10 bp, Homo sapiens
taagtgtggt

SEQ ID NO: 83 - DNA, 10 bp, Homo sapiens
ccagcttcct

SEQ ID NO: 84 - DNA, 10 bp, Homo sapiens
agtatctggg

SEQ ID NO: 85 - DNA, 10 bp, Homo sapiens
aagttgctat

SEQ ID NO: 86 - DNA, 10 bp, Homo sapiens
tagcagcaat

SEQ ID NO: 87 - DNA, 10 bp, Homo sapiens
tatgaatgct

SEQ ID NO: 88 - DNA, 10 bp, Homo sapiens
gtcttaaagt

SEQ ID NO: 89 - DNA, 10 bp, Homo sapiens
agatgagatg

SEQ ID NO: 90 - DNA, 10 bp, Homo sapiens
gaaaagtttc

SEQ ID NO: 91 - DNA, 10 bp, Homo sapiens
ttggtttttg

SEQ ID NO: 92 - DNA, 10 bp, Homo sapiens
ccggccctac

SEQ ID NO: 93 - DNA, 10 bp, Homo sapiens
cgcccgtcgt

SEQ ID NO: 94 - DNA, 10 bp, Homo sapiens
gaaaaataca

SEQ ID NO: 95 - DNA, 10 bp, Homo sapiens
acagaaggga

SEQ ID NO: 96 - DNA, 10 bp, Homo sapiens
tgccctcaaa

SEQ ID NO: 97 - DNA, 10 bp, Homo sapiens
tgccctcagg

SEQ ID NO: 98 - DNA, 10 bp, Homo sapiens
gggattaaag

SEQ ID NO: 99 - DNA, 10 bp, Homo sapiens
ttctatttca

SEQ ID NO: 100 - DNA, 10 bp, Homo sapiens
cctgaggaat

SEQ ID NO: 101 - DNA, 10 bp, Homo sapiens
ttgctgccag

SEQ ID NO: 102 - DNA, 10 bp, Homo sapiens
gtcgaaggac

SEQ ID NO: 103 - DNA, 10 bp, Homo sapiens
gtgccggagg

SEQ ID NO: 104 - DNA, 10 bp, Homo sapiens
gtgcgaagga

SEQ ID NO: 105 - DNA, 10 bp, Homo sapiens
tcgctgcttt

SEQ ID NO: 106 - DNA, 10 bp, Homo sapiens
tggtgttaag

SEQ ID NO: 107 - DNA, 10 bp, Homo sapiens
cctatgtaag

SEQ ID NO: 108 - DNA, 10 bp, Homo sapiens
ggaaaagtgg

SEQ ID NO: 109 - DNA, 10 bp, Homo sapiens
gtgcggagga

SEQ ID NO: 110 - DNA, 10 bp, Homo sapiens
ttgggggttt

SEQ ID NO: 111 - DNA, 10 bp, Homo sapiens
tctgcaaatt

SEQ ID NO: 112 - DNA, 10 bp, Homo sapiens
tcaggcctgt

SEQ ID NO: 113 - DNA, 10 bp, Homo sapiens
gaaaagtttc

SEQ ID NO: 114 - DNA, 10 bp, Homo sapiens
ttgaaacttt

SEQ ID NO: 115 - DNA, 10 bp, Homo sapiens
tggaagcact

SEQ ID NO: 116 - DNA, 10 bp, Homo sapiens
ggaggtaggg

SEQ ID NO: 117 - DNA, 10 bp, Homo sapiens
ctgtgagacc

SEQ ID NO: 118 - DNA, 10 bp, Homo sapiens
tggtccagcg

SEQ ID NO: 119 - DNA, 10 bp, Homo sapiens
gttcacatta

SEQ ID NO: 120 - DNA, 10 bp, Homo sapiens
tgactggcag

SEQ ID NO: 121 - DNA, 10 bp, Homo sapiens
gcagttctga

SEQ ID NO: 122 - DNA, 10 bp, Homo sapiens
acagaaggga

SEQ ID NO: 123 - DNA, 10 bp, Homo sapiens
atagctgggg

SEQ ID NO: 124 - DNA, 10 bp, Homo sapiens
actgaggaaa

SEQ ID NO: 125 - DNA, 10 bp, Homo sapiens
atcaaatgca

SEQ ID NO: 126 - DNA, 10 bp, Homo sapiens
ggaggtaggg

SEQ ID NO: 127 - DNA, 10 bp, Homo sapiens
ggatgcaagg

SEQ ID NO: 128 - DNA, 10 bp, Homo sapiens
gacctcctgc

SEQ ID NO: 129 - DNA, 10 bp, Homo sapiens
ttctatttca

SEQ ID NO: 130 - DNA, 10 bp, Homo sapiens
agtatctggg

SEQ ID NO: 131 - DNA, 10 bp, Homo sapiens
ctggcgcgag

SEQ ID NO: 132 - DNA, 10 bp, Homo sapiens
acagagcaca

SEQ ID NO: 133 - DNA, 10 bp, Homo sapiens
tgcagtcact

SEQ ID NO: 134 - DNA, 10 bp, Homo sapiens
ggaaaagtgg

SEQ ID NO: 135 - DNA, 10 bp, Homo sapiens
tttccctcaa

SEQ ID NO: 136 - DNA, 10 bp, Homo sapiens
ttgatgcccg

SEQ ID NO: 137 - DNA, 10 bp, Homo sapiens
tatgacttaa

SEQ ID NO: 138 - DNA, 10 bp, Homo sapiens
aggtttcctc

SEQ ID NO: 139 - DNA, 10 bp, Homo sapiens
atgggatggc

SEQ ID NO: 140 - DNA, 10 bp, Homo sapiens
cccaacgcgc

SEQ ID NO: 141 - DNA, 10 bp, Homo sapiens
cccgaggcag

SEQ ID NO: 142 - DNA, 10 bp, Homo sapiens
ctttgagtcc

SEQ ID NO: 143 - DNA, 10 bp, Homo sapiens
gatgcgagga

SEQ ID NO: 144 - DNA, 10 bp, Homo sapiens
gcaagaaagt

SEQ ID NO: 145 - DNA, 10 bp, Homo sapiens
gcaggccaag

SEQ ID NO: 146 - DNA, 10 bp, Homo sapiens
gccttccaat

SEQ ID NO: 147 - DNA, 10 bp, Homo sapiens
gggattaaag

SEQ ID NO: 148 - DNA, 10 bp, Homo sapiens
gtaatgacag

SEQ ID NO: 149 - DNA, 10 bp, Homo sapiens
gtctggggga

SEQ ID NO: 150 - DNA, 10 bp, Homo sapiens
gtgcggagga

SEQ ID NO: 151 - DNA, 10 bp, Homo sapiens
gtggtggaca

SEQ ID NO: 152 - DNA, 10 bp, Homo sapiens
gtgtctcgca

SEQ ID NO: 153 - DNA, 10 bp, Homo sapiens
tggaaagctt

SEQ ID NO: 154 - DNA, 10 bp, Homo sapiens
tggcttgctc

SEQ ID NO: 155 - DNA, 10 bp, Homo sapiens
ttgaatcccc

SEQ ID NO: 156 - DNA, 10 bp, Homo sapiens
gacggcgcag

SEQ ID NO: 157 - DNA, 10 bp, Homo sapiens
tttgcacctt

SEQ ID NO: 158 - DNA, 10 bp, Homo sapiens
gaaaagtttc

SEQ ID NO: 159 - DNA, 10 bp, Homo sapiens
ttgaaacttt

SEQ ID NO: 160 - DNA, 10 bp, Homo sapiens
aagattgggg

SEQ ID NO: 161 - DNA, 10 bp, Homo sapiens
gtacggagat

SEQ ID NO: 162 - DNA, 10 bp, Homo sapiens
ttcaggaggg

SEQ ID NO: 163 - DNA, 10 bp, Homo sapiens
tcgaagaacc

SEQ ID NO: 164 - DNA, 10 bp, Homo sapiens
aaaactgaga

SEQ ID NO: 165 - DNA, 10 bp, Homo sapiens
acagaaggga

SEQ ID NO: 166 - DNA, 10 bp, Homo sapiens
ccaggctgcg

SEQ ID NO: 167 - DNA, 10 bp, Homo sapiens
gtactgtagc

SEQ ID NO: 168 - DNA, 10 bp, Homo sapiens
atcaaatgca

SEQ ID NO: 169 - DNA, 10 bp, Homo sapiens
ggagggatca

SEQ ID NO: 170 - DNA, 10 bp, Homo sapiens
atggccatag

SEQ ID NO: 171 - DNA, 10 bp, Homo sapiens
cagcgccacc

SEQ ID NO: 172 - DNA, 10 bp, Homo sapiens
accatcctgc

SEQ ID NO: 173 - DNA, 10 bp, Homo sapiens
aaagtctaga

SEQ ID NO: 174 - DNA, 10 bp, Homo sapiens
ttccactaac

SEQ ID NO: 175 - DNA, 10 bp, Homo sapiens
ttctatttca

SEQ ID NO: 176 - DNA, 10 bp, Homo sapiens
agtatctggg

SEQ ID NO: 177 - DNA, 10 bp, Homo sapiens
atcttgttac

SEQ ID NO: 178 - DNA, 10 bp, Homo sapiens
ggaaaagtgg

SEQ ID NO: 179 - DNA, 10 bp, Homo sapiens
gcacctgtcg

SEQ ID NO: 180 - DNA, 10 bp, Homo sapiens
gcaaaaaaaa

SEQ ID NO: 181 - DNA, 10 bp, Homo sapiens
gatcctggat

SEQ ID NO: 182 - DNA, 10 bp, Homo sapiens
ttcactgccg

SEQ ID NO: 183 - DNA, 10 bp, Homo sapiens
acaaaccccc

SEQ ID NO: 184 - DNA, 10 bp, Homo sapiens
cacagtcaaa

SEQ ID NO: 185 - DNA, 10 bp, Homo sapiens
aagcaggagg

SEQ ID NO: 186 - DNA, 10 bp, Homo sapiens
aatgcttgat

SEQ ID NO: 187 - DNA, 10 bp, Homo sapiens
acaaatcctt

SEQ ID NO: 188 - DNA, 10 bp, Homo sapiens
actcagcccg

SEQ ID NO: 189 - DNA, 10 bp, Homo sapiens
cacacccctg

SEQ ID NO: 190 - DNA, 10 bp, Homo sapiens
ccaggggaga

SEQ ID NO: 191 - DNA, 10 bp, Homo sapiens
cgaccccacg

SEQ ID NO: 192 - DNA, 10 bp, Homo sapiens
gctgcccggc

SEQ ID NO: 193 - DNA, 10 bp, Homo sapiens
taaaaatgtt